(19) United States
(12) Patent Application Publication
(10) Pub. No.: US 2024/0401143 A1
(43) Pub. Date: Dec. 5, 2024

(54) AQUEOUS HUMOR CELL-FREE DNA AND OPHTHALMIC DISEASE

(71) Applicants: Children's Hospital Los Angeles, Los Angeles, CA (US); University of Southern California, Los Angeles, CA (US)

(72) Inventors: Jesse Lee Berry, Pasadena, CA (US); James Bruce Hicks, Palm Desert, CA (US); Liya Xu, La Canada Flintridge, CA (US)

(21) Appl. No.: 17/045,435

(22) PCT Filed: Apr. 6, 2019
(86) PCT No.: PCT/US2019/026221

§ 371 Date: Oct. 5, 2020

Related U.S. Application Data
(60) Provisional application No. 62/654,160, filed on Apr. 6, 2018.

(51) Int. Cl. C12Q 1/6886

(52) U.S. Cl. CPC C12Q 1/6886; C12Q 2600/118; C12Q 2600/156; C12Q 2600/178

(57) ABSTRACT

Tumor-derived cell-free DNA from the aqueous humor is assayed for diagnosis, and therapeutic prognosis of retinoblastoma demonstrating eyes with a more aggressive phenotype (with the presence of gain of 6p) and those that have a higher likelihood of responding to therapy. This also allows for evaluation of tumor response to therapy and a marker of recurrent or residual disease that requires further therapy. This method avoids direct biopsy of retinoblastoma, which is contraindicated due to risk of extraocular tumor dissemination. Somatic chromosomal copy number alterations of tumor-derived cell-free DNA provides significant prognostic value; the presence of gain of 6p was associated with a 10× increased risk of a tumor NOT responding to chemotherapy with local consolidation and requiring enucleation to cure the child of disease. AH testing also provides accurate indicators of progression of retinoblastoma and its response to therapy over the course of treatment. In various embodiments, a gain in the somatic chromosomal copy number of chromosome 6p from aqueous humor is highly correlated with severity of retinoblastoma to indicate enucleation as a potential required intervention to cure disease.

### CLAIM OF PRIORITY

This application claims the benefit of priority of U.S. Provisional Patent Application No. 62/654,160, filed on Apr. 6, 2018, the benefit of priority of which is claimed hereby, and which is incorporated by reference herein in its entirety.

### GOVERNMENT SUPPORT

This invention was made with Government support under Grant (or Contract) No. K08CA232344, awarded by the National Institutes of Health (NIH). The Government has certain rights in this invention.

### BACKGROUND

Retinoblastoma is a primary cancer that develops in the eyes of children. While various treatment modalities exist, enucleation, or surgical removal of the entire eye, is still needed for advanced tumors (1,2). Primary enucleation is performed when the tumor appears to be too advanced for attempted salvage therapy. Secondary enucleation is required when the tumor recurs after chemotherapy and the eye is removed to prevent tumor spread. Currently, prediction of which eyes will respond to therapy (and avoid enucleation) is based on clinical classifications which include tumor size, retinal detachment and tumor seeding (3). However, the most commonly used classification, the International Intraocular Retinoblastoma Classification (IIRC) (3), is predictive of treatment success in only 50% of Group D eyes (4,5) and is even less predictive for more advanced Group E eyes (6,7).

A notable difference in the diagnostic classification of retinoblastoma compared to other cancers, is that it is not based on biopsy and does not consider any genetic tumor markers (8). Nonetheless, much is known about retinoblastoma genetics from studies of tumor tissue from enucleated eyes. The vast majority of retinoblastoma (98%) is initiated by inactivation of both alleles of the RB1 tumor suppressor gene on chromosome 13q (9-13). Additional genetic changes can further drive tumorigenesis (14,15). Tumor studies have revealed somatic copy number alteration (SCNA) profiles with highly recurrent chromosomal gains on 1q, 2p, 6p, losses on 13q, 16q, and focal MYCN amplification on 2p which together are termed ‘RB SCNAs’ (9,10,12,13).

The role RB SCNAs play in retinoblastoma tumorigenesis and moreover, whether there are certain SCNAs that portend a more aggressive tumor phenotype, is unknown. One report Indicates that 1q and 6p gain and 16q loss may be associated with locally invasive disease (16); another suggests gain of 6p is associated with less differentiated tumors with higher rates of optic nerve invasion (17) and may be seen in older patients (18). However, these associations have not been relevant to predicting eye salvage, nor applied to tumors at diagnosis or during therapy. This is because invasive tissue biopsy of retinoblastoma is contraindicated due to reports of extraocular tumor spread after biopsy, which significantly changes the prognosis for the child (19,20).

### SUMMARY OF THE INVENTION

The following embodiments and aspects thereof are described and illustrated in conjunction with compositions and methods which are meant to be exemplary and illustrative, not limiting in scope.

Retinoblastoma cannot be biopsied directly due to the risk of extraocular tumor dissemination, thus until recently, neither tumor derived DNA nor any other tumor biomarker was used for diagnosis, prognosis or disease management. Applicant demonstrated that tumor-derived cell-free DNA (cfDNA) is present in the aqueous humor (21), which is the clear fluid in a separate compartment of the eye from where the tumor forms and can be safely sampled, at diagnosis and longitudinally throughout treatment, without fear of tumor spread (22). Thus, for the first time, tumor DNA is accessible during treatment of eyes with retinoblastoma whereas previously it was only available after the eye had been surgically removed (enucleated).

Therefore, it is an objective of the present invention to provide biomarkers easily accessible without excessive damage to the eye or risk of extraocular tumor spread for evaluation of tumor response to therapy, therapeutic intervention, and eye salvage to avoid enucleation.

It is another objective of the present invention to provide a process of performing diagnosis and/or prognosis of eye tumor response to therapy, therapeutic intervention, and eye salvage using an easily accessible specimen for accurate analysis.

In various embodiments, tumor-derived cell-free DNA (cfDNA) is identified in the aqueous humor (AH) of retinoblastoma eyes. Somatic chromosomal copy number alterations (SCNAs) in the AH are correlated with clinical outcomes, specifically eye salvage (e.g., the ability to cure the intraocular cancer and save the eye).

In various embodiments of the invention, gain of chromosome 6p is associated with a 10× increased odds of an eye failing treatment and ultimately requiring an enucleation (surgical removal of the eye).

Other features and advantages of the invention will become apparent from the following detailed description, taken in conjunction with the accompanying drawings, which illustrate, by way of example, various features of embodiments of the invention.

In some embodiments, AH can be used to test residual active disease which can be used by the clinician to continue or activate further therapy before the disease advances and/or becomes resistant to current therapy.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1A-1G depict Chromosomal Copy Number Alteration (CNA) profiles from 7 eyes that required enucleation with available tumor tissue for comparison. The profiles demonstrate the similar genomic alteration between the AH and tumor, with the notable exception of Case 1 (A) which is due to the presence of multiple intraocular tumors.

FIG. 2 depicts Pearson's hierarchical clustering matrix based on the SCNA profiles of the 58 AH and tumor samples from 21 eyes that had more than one sample available for correlation. Samples are listed as Case number_# based on the chronological order of AH sampling (e.g. 1, 2, 3) with longitudinal AH samples designated by a hyphen followed by sample number (e.g. 1-1, 1-2, 2-1). Tumor samples correlate most closely with the matched AH samples from the same eye (with the notable exception of Case 1, described in text, with multiple intraocular tumors). The majority of longitudinal AH samples also group together with few exceptions. Samples that correlate within the same eye are shown by the grey bars on the right, the black bars indicate samples that did not fall adjacent other samples from the same eye. Samples from eyes that were enucleated (e.g. surgically removed) are indicated by the red bar adjacent the dentogram, those that are salvaged (e.g. saved) are indicated in blue. This shows that aqueous humor samples from the same correlate together and are consistent longitudinally.

FIG. 3A depicts composite somatic copy number alteration (CNA) profile from cell-free DNA in the Aqueous Humor (AH) samples from enucleated eyes (Enuc, red) and salvaged eyes (Salv, blue). FIG. 3B depicts a box plot demonstrating the range of amplitude changes for the enucleated (Enuc) vs. salvaged (Salv) eyes; the black bar represents the median while the green bar represents the mean (of the ratio to median). The sample with focal MYCN gain is shown as a red asterisk in the Chr 2p plot. The mean of the ratio to median amplitude of Chr 6p gain is significantly greater in enucleated eyes (p=0.001), which is both from the increased copy number of the amplified region and an increase in the total fraction of tumor-derived DNA in the AH of enucleated eyes.

FIGS. 4A-4F depict Kaplan-Meier curves of eye salvage/survival for treated eyes (e.g. no primary enucleations) at 800 days by (4A) all eyes and all risk groups (with time from diagnosis to event or last follow-up); (4B) all eyes+/−presence of genomic instability >300 sum deviation from the median (with time from sample to event or last follow-up), regardless of clinical staging; (4C) all eyes+/−the presence of RB SCNAs in the AH (with time from sample to event or last follow-up), regardless of clinical staging; (4D) all eyes+/−presence of gain of 6p in the AH (with time from sample to event or last follow-up), regardless of clinical staging; (4E) Group D eyes+/−the presence of RB SCNAs in the AH (with time from sample to event or last follow-up); (4F) Group E eyes+/−the presence of RB SCNAs in the AH (with time from sample to event or last follow-up).

This demonstrates that the presence of any RB SCNA aids in prediction of globe salvage more accurately than Group Classification alone. Within the RB SCNAs, 6p gain was most predictive of risk of tumor recurrence requiring enucleation.

FIGS. 5A and 5B depict Copy Number Alteration (CNA) profile and histogram from two cases demonstrating changes in amplitude of alterations that correlate with clinical tumor response. The CNA profiles for Case 6 (FIG. 5A) demonstrates increased chromosomal alterations in chromosomes 1q, 2p, 6p and 16q; additionally, 7q, 11q and 12q were altered and are shown. AH samples 1-5 were taken longitudinally separated by at least 1 week between sample. Case 6 demonstrates decreased CNA magnitude at AH sample 2 relative to sample 1 which correlated with clinical response to therapy; however, these alterations then increase steadily with persistent tumor activity and this eye eventually required enucleation. The CNA profile from the tumor (shown in FIG. 1) mimics the AH profile. Case 22 (FIG. 5B) demonstrates an opposite finding: as the tumor responded to therapy the CNA magnitude from the AH declined. This suggest both a smaller concentration of tumor-derived DNA and a more stable tumor genomic state, as represent by the AH, as the tumor responds to therapy.

FIG. 6 demonstrates representative profiles from the AH and the blood in a patient with retinoblastoma in both the right (blue) and left eye (green). While the AH demonstrated copy number alterations in both eyes (which differ, due to differential modes of tumorigenesis), the blood (red) does not show copy number alterations.

FIG. 7 demonstrates representative profiles from the AH (blue) and the blood (red) from 3 additional patients again demonstrating the presence of copy number alterations in the AH ONLY and not in the blood.

FIG. 8 shows the peak cell-free DNA fragment size in the AH (blue, green for second eye) vs the blood (red).

FIG. 9 provides a graphic summarizing data from multiple studies on miRNA in retinoblastoma tumor.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application. For references on how to prepare antibodies, see D. Lane, Antibodies: A Laboratory Manual 2^{nd} ed. (Cold Spring Harbor Press, Cold Spring Harbor NY, 2013); Kohler and Milstein, (1976) Eur. J. Immunol. 6: 511; Queen et al. U.S. Pat. No. 5,585,089; and Riechmann et al., Nature 332: 323 (1988); U.S. Pat. No. 4,946,778; Bird, Science 242:423-42 (1988); Huston et al., Proc. Natl. Acad. Sci. USA 85:5879-5883 (1988); Ward et al., Nature 334:544-54 (1989); Tomlinson I. and Holliger P. (2000) Methods Enzymol, 326, 461-479; Holliger P. (2005) Nat. Biotechnol. September; 23(9):1126-36).

Genomic analysis of the AH samples is provided which reproducibly reflects the genomic state of the tumor and the highly recurrent RB SCNAs detected in the AH are shown to be predictable of tumor response to therapy. Applicant performed genomic evaluation for copy number alterations and correlated these tumor biomarkers with therapeutic tumor response and the ability to salvage the eye. In various embodiments of the invention, gain of chromosome 6p is associated with a 10× increased odds of an eye failing treatment and ultimately requiring an enucleation (surgical removal of the eye). Because tumor DNA has never been previously available in eyes prior to enucleation, this is the first time a clinical biomarker has been demonstrated.

Shallow or low-pass whole genome sequencing is used when no full genome coverage is needed. This technique can be used for detection of aneuploidy and/or chromosomal imbalances.

### Treatment of Retinoblastoma

The priority of retinoblastoma treatment is to preserve the life of the child, then to preserve the eye, then to preserve vision, all while minimizing complications or side effects of treatment. The exact course of treatment will depend on the individual case, whether one or both eyes are affected with the cancer, and will be decided by the ophthalmologist in discussion with the pediatric oncologist. Children with involvement of both eyes at diagnosis usually require multimodality therapy (chemotherapy, local therapies).

The various treatment modalities for retinoblastoma includes:

Enucleation of the eye—Most patients with unilateral disease present with advanced intraocular disease and therefore often undergo enucleation, which results in a cure rate of 95%. In bilateral Rb, enucleation is usually reserved for eyes that have failed all known effective therapies or without useful vision.

External beam radiotherapy (EBR)—The most common indication for EBR is for the only remaining eye in a young child with bilateral retinoblastoma who has active or recurrent disease after completion of chemotherapy and local therapies. However, patients with hereditary disease who received EBR therapy are reported to have a 35% risk of second cancers.

Brachytherapy—Brachytherapy involves the placement of a radioactive implant (plaque), on the sclera adjacent to the base of a tumor. It used as the primary treatment in medium sized <6 mm tumors without diffuse seeding or, more frequently, in patients with recurrent tumors after failing initial therapy including systemic chemotherapy, intra-arterial chemotherapy and local consolidation.

Thermotherapy—Thermotherapy involves the application of heat directly to the tumor, usually in the form of infrared radiation via a red diode laser. It is used to consolidate residual active disease after chemotherapy and also used as primary therapy for very small tumors (<3 mm).

Laser photocoagulation—Laser photocoagulation is recommended as primary therapy only for small posterior tumors, it is standard to treat residual active disease after chemotherapy with green and/or red (argon/diode) laser. This is called consolidation. An argon or diode laser or a xenon arc is used to coagulate all the blood supply to the tumor.

Cryotherapy—Cryotherapy induces damage to the vascular endothelium with secondary thrombosis and infarction of the tumor tissue by rapidly freezing it. Cryotherapy may be used as primary therapy for small peripheral tumors or for small recurrent tumors previously treated with other methods.

Systemic chemotherapy—Systemic chemotherapy most frequently with a 3-drug regimen has been used as for the past several decades as treatment for retinoblastoma as a globe preserving measure and to avoid the adverse effects of EBR therapy. The common indications for chemotherapy for intraocular retinoblastoma include tumors that are large and that cannot be treated with local therapies alone in children with bilateral tumors. It is also used in patients with unilateral disease when the tumors are not so advanced to have destroyed all intraocular structures (eg Group E eyes) but cannot be controlled with local therapies alone (Group B-D eyes).

Intra-arterial chemotherapy—Chemotherapeutic drugs are administered locally via a thin catheter threaded through the groin, through the aorta and the neck, directly into the optic vessels. This is generally reserved for advanced unilateral retinoblastoma (Group C or D) however has been used in ‘tandem’ for bilateral disease at some centers.

Nano-particulate chemotherapy—To reduce the adverse effects of systemic therapy, subconjuctival (local) injection ofnanoparticle carriers containing chemotherapeutic agents (carboplatin) has been developed which has shown promising results in the treatment of retinoblastoma in animal models without adverse effects.

Chemoreduction—A combined approach using chemotherapy to initially reduce the size of the tumor, and adjuvant focal treatments, such as transpupillary thermotherapy, to control the tumor.

Standard therapy is generally either systemic or intra-arterial chemotherapy, depending on the stage and laterality of disease, with consolidation that may include laser therapy, thermotherapy, cryotherapy or rarely brachytherapy. External Beam radiation is generally avoided. High-dose chemotherapy with bone marrow transplant is not done for intraocular retinoblastoma, it is indicated for extraocular or metastatic disease. Intravitreal injection of chemotherapy is done with a 32-gauge needle via the pars plana via the sclera. Most frequently melphalan or topotecan are injected directly into the posterior segment of the eye. This therapy is indicated for the treatment of vitreous seeds (small floating pieces of viable retinoblastoma tumor in the vitreous cavity).

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Experimental Design

AH was extracted via paracentesis during intravitreal injection of chemotherapy or enucleation. CfDNA was isolated; shallow whole genome sequencing performed to assess tumor DNA fractions and known, highly recurrent SCNAs in retinoblastoma including gain of 1q, 2p, 6p, loss of 13q, 16q and focal MYCN amplification. Age at diagnosis, clinical classification, treatment regimen and eye salvage were recorded. Clinical analysis was retrospective.

### Overall Results

Sixty-three samples of AH from 29 eyes of 26 patients were evaluated. Ultimately 13 eyes required enucleation and 16 were salvaged. The presence of detectable SCNAs was 92% in enucleated eyes versus 38% in salvaged eyes (p=0.006). 6p gain was the most common SCNA found in 77% of enucleated eyes versus 25% of salvaged eyes (p=0.0092). 6p gain was associated with a ten-fold increased odd of enucleation (OR=10, 95% CI:1.8-55.6). The mean amplitude of 6p gain was 1.47 in enucleated eyes versus 1.07 in salvaged eyes (p=0.001). The probability of ocular survival was higher in eyes without detectable SCNAs in the AH (p=0.0028).

### SUMMARY

To the best of Applicant's knowledge, this is the first study to show that clinical outcomes correlate with highly-recurrent SCNAs in the AH from retinoblastoma eyes. This study shows that the AH can reliably serve as a surrogate to tumor biopsy and genomic analysis improves upon clinical staging to predict the ability to salvage the eye.

### Detailed Methods

Institutional Review Board approval was obtained with written informed consent from the parents of participants. Samples were sequenced within one month of extraction. Genomic data was kept separate from the clinical data until analysis, which was done retrospectively. REMARK guidelines for reporting biomarkers were followed.

### Surgical Procedure

Clear corneal paracentesis was performed with extraction of 0.1 ml of AH as part of the procedure for intravitreal injection of chemotherapy (23) or immediately post-enucleation. Samples were stored at −80° C. CfDNA isolation and sequencing protocols were described previously (21).

### Data Analysis

SCNA analyses were described previously (21,24,25). Next Generation Sequencing reads from pooled barcoded DNA libraries were deconvoluted (Illumina iGenome) and mapped to the human genome (hg19, Genome Reference Consortium GRCh37(26)) with Bowtie2 (27,28). Duplicates were removed (samtools rmdup(29)), normalized for G:C content, and DNA segment copy numbers obtained by dividing the genome into 5000 variable length bins and calculating the relative number of reads in each bin. Copy number estimates were calculated by reference-free log 2-ratios taking the median window count of normal autosomal chromosomes. Segmentation was performed using circular binary segmentation with DNA copy (Bioconductor (30)). SCNAs were positive at 20% deflection from baseline (log 2-ratio=0) meaning losses at log 2-ratios <−0.2 (ratio of 0.87 or lower) and gains at log 2-ratios >0.2 (ratio of 1.15 or higher). Hierarchical clustering was performed using heatmap.2 function in R package gplots on median centered data, using Ward's method (31,32) as the distance metric. Clustering was based on Pearson correlation of the SCNA profiles.

Genomic instability was calculated as the sum of the segmented log 2-ratios, excluding chromosome X and Y and represented as the sum deviation from the median. AH samples with <2% of reads aligned to the human genome were removed from analysis.

Kaplan-Meier survival analyses with log-rank tests compared eye salvage in treated eyes based on IIRC groups (3), and presence of RB SCNAs. A mixed model test compared mean amplitudes of 6p gain in enucleated versus salvaged eyes, accounting for biological replicates and within-patient variations by eye. Fisher's exact tests were used for associations between presence of RB SCNAs and clinical classification, or outcome. JMP Pro 13 (Cary, NC, USA) was used for statistical analyses.

Charts were reviewed for age at diagnosis, sex, laterality, IIRC group (3), treatment modalities, tumor recurrence, enucleation, and follow-up.

### Detailed Results

To assess relationships between AH and SCNAs, a data set was assembled including sequential AH samples, matched tumors from enucleated eyes and clinical outcomes. Demographics of the 26 patients are in Table 1; three patients had both eyes included for a total of 29 eyes. Thirteen eyes required enucleation (3 primarily and 10 secondarily due to tumor relapse); 16 were salvaged with treatment. Clinical follow-up ranged 8-43 months (median 17 months).

### Table 1

Table 1 provides Patient Demographics, Clinical Outcomes and RB SCNA genomic alterations: Eyes that required enucleation are above the grey line and those that were salvaged are below. Gains or losses are indicated as gain; loss, along with amplitude of the change (as ratio to median). Notes: AH=aqueous humor; CEV=carboplatin, etoposide, vincristine systemic chemotherapy; ENUC=enucleation; mos=months; mtn=mutation; RB=retinoblastoma; RB1=retinoblastoma tumor suppressor gene; SCNA=somatic copy number alteration; Tx=therapy; +=SCNA not present in the initial AH sample, but present in subsequent (Case 11); *=SCNA present in the initial AH sample but NOT present in subsequent (Cases 21, 25); **=SCNA not present in initial AH sample however required secondary enucleation for a late (>800 days) massive retinal recurrence and AH was not taken at that time (Case 5).

| | | | IIRC | | blood | | Timing | Total # | 1 |
|---|---|---|---|---|---|---|---|---|---|
| | | Age at | Group | Later- | RB1 | Initial | of 1^{st} | of AH | [symbol] |
| Case | Sex | Dx (mos) | Class | ality | mutation | Tx | AH Sample | samples | 1q |
| 1 | M | 20 | E | U | 13q- | ENUC | with | 1 | |
| | | | | | | | enuc | | |
| 2 | M | 7 | E | U | negative | ENUC | with | 1 | |
| | | | | | | | enuc | | |
| 3 | M | 38 | E | U | negative | ENUC | with | 1 | ↑(1.3) |
| | | | | | | | enuc | | |
| 4 | M | 26 | D | U | negative | CEV | IVM | 2 | ↑(1.4) |
| 5 | M | 0 | B | B | g.76932_76952del21 | CEV | IVM | 1 | |
| 6 | F | 28 | D | B | 30% mosaic | CEV | IVM | 7 | ↑(1.5) |
| | | | | | c.1075 > T | | | | |
| 7 | M | 10 | E | B | frameshift | CEV | IVM | 1 | |
| | | | | | mtn exon 18 | | | | |
| 8 | F | 22 | D | U | negative | CEV | IVM | 3 | ↑(1.1) |
| 9 | F | 29 | D | B | c.958C > | CEV | with | 1 | ↑(1.7) |
| | | | | | T Exon 10 | | enuc | | |
| 10 | F | 2 | E | B | c.2425delC | CEV | with | 1 | |
| | | | | | | | enuc | | |
| 11 | F | 8 | D | U | negative | CEV | IVM | 4 | |
| 12_OS | F | 13 | D | B | 13q & 16p | CEV | IVM | 1 | |
| | | | | | deletion | | | | |
| 13 | M | 34 | D | U | p.Met148 | CEV | IVM | 2 | ↑(1.2) |
| | | | | | Valf | | | | |
| 12_OD | F | 13 | C | B | 13q & 16p | CEV | IVM | 3 | ↑(1.3) |
| | | | | | deletion | | | | |
| 14 | M | 5 | D | U | negative | CEV | IVM | 1 | |
| 15 | M | 10 | D | U | negative | IAM | IVM | 3 | |
| 16_OD | M | 9 | E | B | c.2527delG(Exon | CEV | IVM | 2 | |
| | | | | | 25) | | | | |
| 16_OS | M | 9 | E | B | c.2527delG(Exon | CEV | IVM | 3 | |
| | | | | | 25) | | | | |
| 17 | F | 2 | E | B | c.1421 + | CEV | IVM | 2 | |
| | | | | | 12_1421 + | | | | |
| | | | | | 32del21bp | | | | |
| 18_OD | M | 26 | C | B | c.2520 + | CEV | IVM | 1 | ↑(2.0) |
| | | | | | 1G > A | | | | |
| | | | | | (intron 24) | | | | |
| 18_OS | M | 26 | D | B | c.2520 + | CEV | IVM | 1 | |
| | | | | | 1G > A | | | | |
| | | | | | (intron 24) | | | | |
| 19 | F | 26 | D | B | c.1981C > | IAM | IVM | 3 | |
| | | | | | T (exon 20) | | | | |
| 20 | F | 2 | D | B | T−>G in | CEV | IVM | 3 | |
| | | | | | exon 17 | | | | |
| 21 | M | 24 | D | B | negative | CEV | IVM | 2 | ↑(1.4) |
| 22 | M | 28 | D | U | c.1981C > | CEV | IVM | 5 | ↑(1.5) |
| | | | | | T (exon 20) | | | | |
| 23 | M | 4 | D | U | negative | CEV | IVM | 1 | |
| 24 | F | 10 | D | U | negative | CEV | IVM | 3 | |
| 25 | F | 59 | D | U | negative | CEV | IVM | 2 | |
| 26 | M | 2 | D | U | negative | CEV | IVM | 2 | |

| | 2 | 6 | 13 | 16 | any | | time to | |
|---|---|---|---|---|---|---|---|---|
| | [symbol] | [symbol] | [symbol] | [symbol] | RB | Req'd | ENUC after | follow-up |
| Case | 2p | 6p | 13q | 16p | SCNA | ENUC? | dx (days) | (mos) |
| 1 | | ↑(2.0) | ↓(0.5) | | yes | yes | 0 | 19 |
| 2 | | ↑(2.6) | | | yes | yes | 14 | 8 |
| 3 | | ↑(1.5) | | ↓(0.5) | yes | yes | 2 | 8 |
| 4 | ↑(1.8) | | | ↓(0.6) | yes | yes | 511 | 17 |
| 5 | | | | | no** | yes | 1166 | 43 |
| 6 | ↑(1.3) | ↑(2.0) | | ↓(0.6) | yes | yes | 537 | 35 |
| 7 | | ↑(1.6) | | | yes | yes | 13 | 31 |
| 8 | | ↑(1.7) | | ↓(0.7) | yes | yes | 257 | 9 |
| 9 | | ↑(1.5) | | | yes | yes | 246 | 16 |
| 10 | Focal | | | | yes | yes | 86 | 10 |
| | MYCN | | | | | | | |
| 11 | | ↑ + (1.5) | | | yes | yes | 426 | 13 |
| 12_OS | | ↑(1.8) | ↓(0.5) | | yes | yes | 300 | 11 |
| 13 | | ↑(1.5) | | ↓(0.7) | yes | yes | 302 | 11 |
| 12_OD | | | ↓(0.5) | ↓(0.7) | yes | no | NA | 11 |
| 14 | | | | | no | no | NA | 26 |
| 15 | | | | | no | no | NA | 11 |
| 16_OD | | | | | no | no | NA | 11 |
| 16_OS | | | | | no | no | NA | 11 |
| 17 | | | | | no | no | NA | 18 |
| 18_OD | | ↑(1.7) | | ↓(0.5) | yes | no | NA | 17 |
| 18_OS | | ↑(1.5) | | ↓(0.5) | yes | no | NA | 17 |
| 19 | | | | | no | no | NA | 18 |
| 20 | | | | | no | no | NA | 26 |
| 21 | | ↑*(1.2) | ↓(0.1) | ↓(0.5) | yes | no | NA | 25 |
| 22 | ↑(1.3) | | | ↓(0.5) | yes | no | NA | 21 |
| 23 | | | | | no | no | NA | 29 |
| 24 | | | | | no | no | NA | 36 |
| 25 | | ↑*(1.2) | ↓(0.6) | | yes | no | NA | 42 |
| 26 | | | | | no | no | NA | 9 |

Genome-wide SCNA profiles were obtained from AH cfDNA by shallow whole-genome sequencing, followed by assigning mapped reads to pre-assigned ‘bins’ across the genome (24,33). Seven tumor and 63 AH samples were included; 5 obtained immediately after enucleation and 58 from 24 eyes undergoing intravitreal injection of chemotherapy. Five of the 63 samples (8%) were removed due to poor read count alignment (<2%). Of the remaining samples, 40 exhibited any SCNA above threshold (69%) and 34 (57%) demonstrated one or more of the highly recurrent ‘RB SCNAs’, namely gains of 1q, 2p, 6p, focal MYCN amplification and losses at 13q and 16q (9,10,13,34) (Table 1). The focus of this analysis is on these RB SCNAs, however, alterations in other chromosomal segments were included when scoring total genomic instability.

a. Genomic Analysis of the AH Demonstrates Similar Profiles to Matched Tumors.

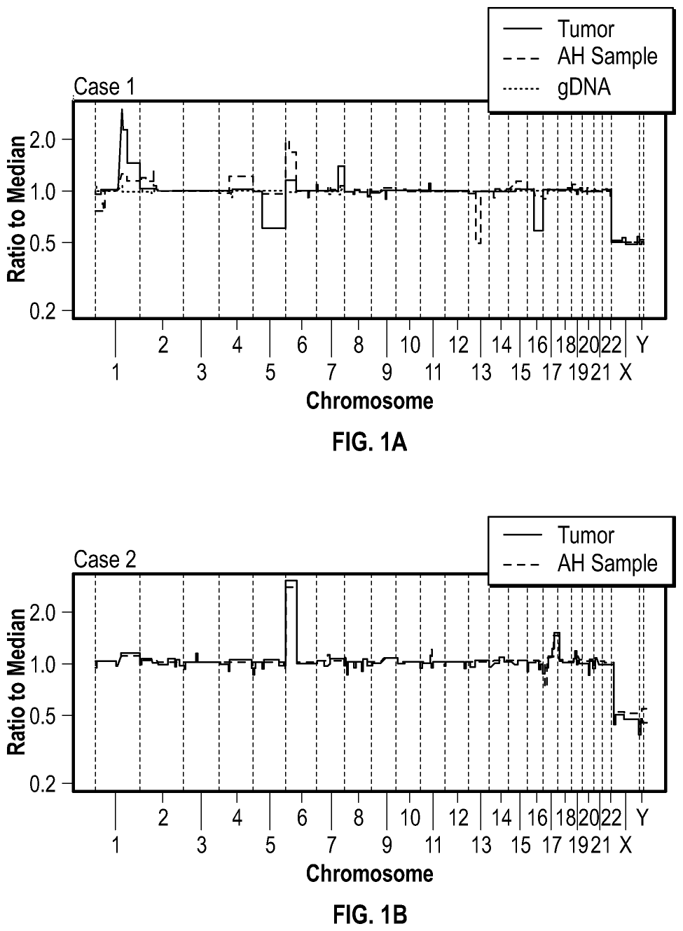

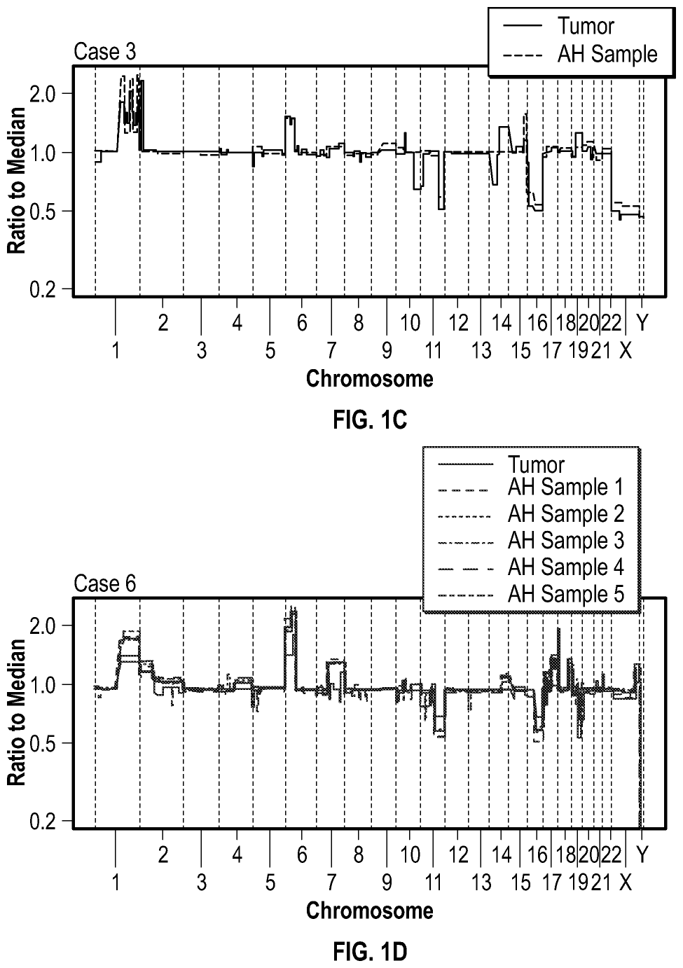

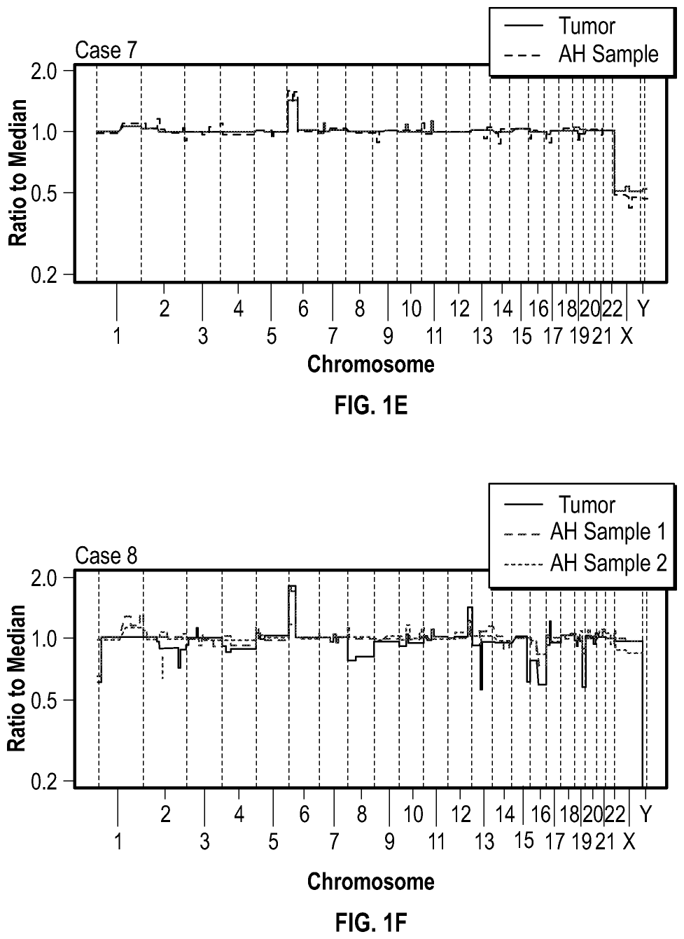

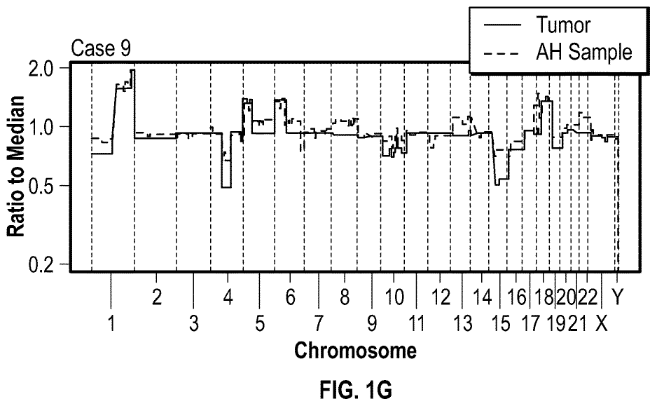

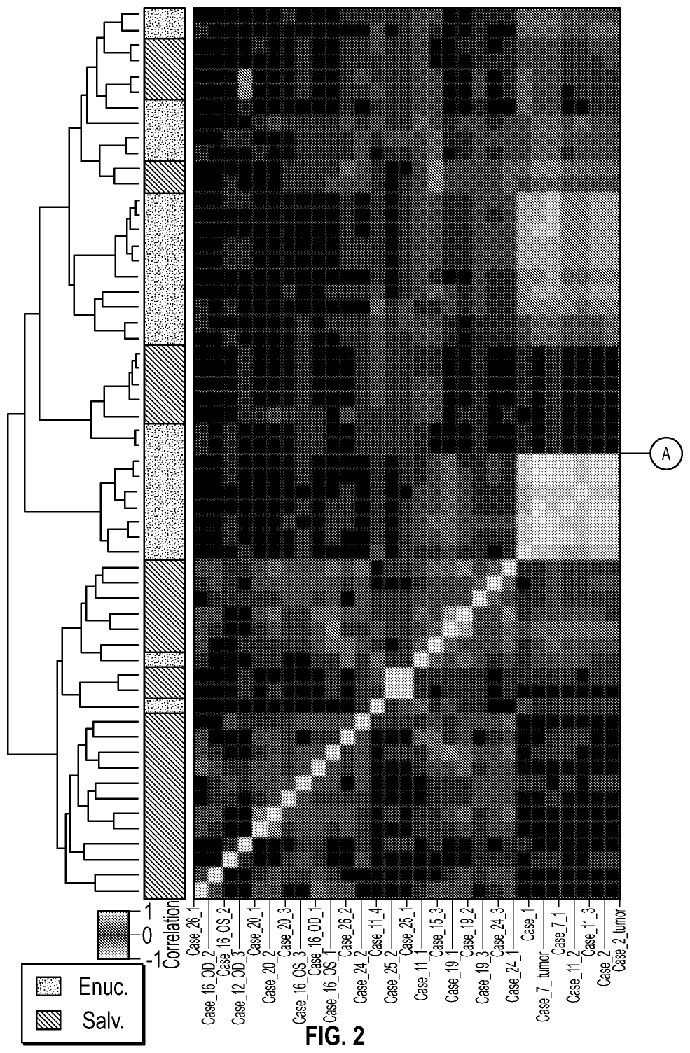

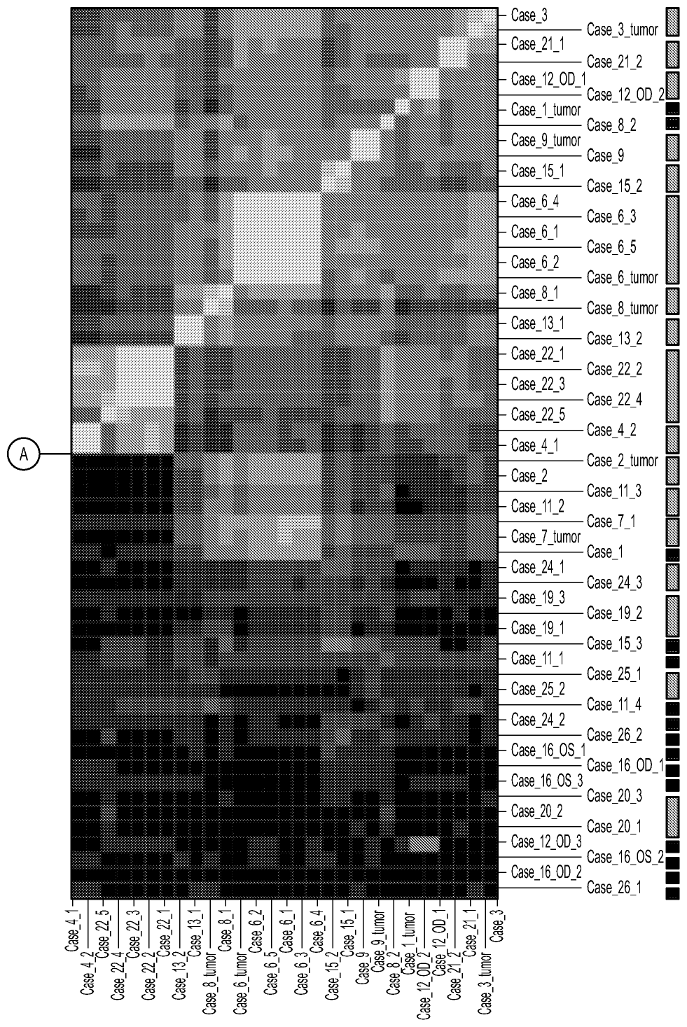

Tumor tissue was available for comparison with AH from 7 enucleated eyes (FIG. 1). Six of these showed a near match of chromosomal gains and losses between tumor and AH, while Case 1 was similar, but the changes did not closely mimic the tumor. This patient had germline loss of a 13q segment predisposing to development of retinoblastoma. This eye (previously described (21)) demonstrated multiple, independent retinal tumors that likely developed different subsets of SCNAs. It was hypothesized that the AH cfDNA profile was likely a heterogeneous mixture of tumor-derived DNA from each separate tumor clone. It was observed that overall the genomic status of the AH matches the genomic status of the tumor, except when multiple retinal tumors were present.

b. Genomic Analysis of AH Samples Longitudinally Demonstrates Reproducibility.

The genomic status of the tumor was evaluated at multiple time points corresponding to sequential intravitreal injections of chemotherapy. To determine whether AH SCNA profiles were stable over time, and correlate with matched tumors, AH and tumor profiles were compared using two different methods. The inter-sample concordance for 58 samples from 21 eyes that had more than 1 sample of AH and/or matched tumor available was compared. FIG. 2 shows a hierarchical clustering matrix (Pearson) containing AH and tumor samples from this subset of samples. Using this method, it was observed that tumor samples correlate most closely with matched AH samples from the same eye (with the exception of Case 1, described above). The majority of longitudinal AH samples also group together with few exceptions such as Case 15 where AH samples 1 and 2 clustered together but sample 3, taken at the last intravitreal injection, had reduced amplitude of alterations and clustered instead with those samples with fewer, lower amplitude aberrations. This demonstrates the high level of inter-sample concordance in the AH with genomic alterations (including low amplitude alterations) remaining stable over sequential draws.

Finally, in agreement with prior analyses of retinoblastoma tumors (9), the overall genomic instability in the AH samples, defined as the total sum deviation from the median of the genome with copy number alterations, positively correlated with age at diagnosis (p=<0.0001, R²=0.658). This observation lends further credence to the hypothesis that the AH is a valid and reliable source of tumor-derived DNA for retinoblastoma.

c. SCNA Profiling of AH cfDNA Revealed Differences in Enucleated and Salvaged Eyes.

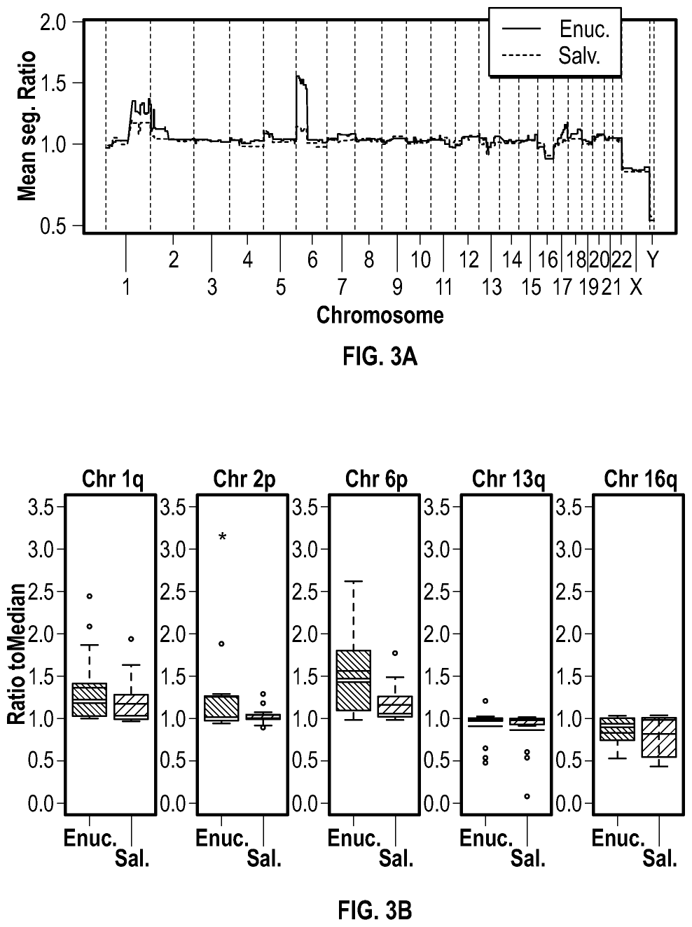

Another goal was to determine whether genomic evaluation of the AH was predictive of eye salvage. The initial AH cfDNA CNA profiles for eyes that had been enucleated were compared against AH profiles for salvaged eyes (FIG. 3). Genomic evaluation of these AH samples revealed that chromosome 6p gains were the most frequent RB SCNA and were significantly more common in enucleated eyes. The 6p gain was present in 10/13 enucleated eyes (77%) as compared to 4/16 (25%) salvaged eyes (Fisher's Exact, p=0.0092). The composite summation of the SCNA profiles from the initial AH samples for the two groups are shown in FIG. 3 revealing the difference in mean amplitude of 6p gain between enucleated and salvaged eyes (1.47 in enucleated eyes versus 1.07 in salvaged eyes, p=0.001). The odds of an eye requiring enucleation were 10 times greater if 6p gain was present in the AH (OR=10; 95% CI: 1.8-55.6).

The presence of any RB SCNA in enucleated eyes was 12/13 (92%) while the fraction in salvaged eyes was 6/16 (38%) (p=0.006). There were no significant differences in 1q, 2p, 13q or 16q between the salvaged and enucleated groups, although there was a marginal effect for 1q (1.22 amplitude gain in enucleated eyes, versus 1.09 gain in salvaged eyes, p=0.08). It is noted that a focal MYCN amplification on 2p without any other SCNAs in the AH of one eye (Case 10).

d. The Presence of RB SCNAs, Specifically Gain of 6p, in the AH Significantly Improves Upon Clinical Staging Alone for Prediction of Eye Salvage.

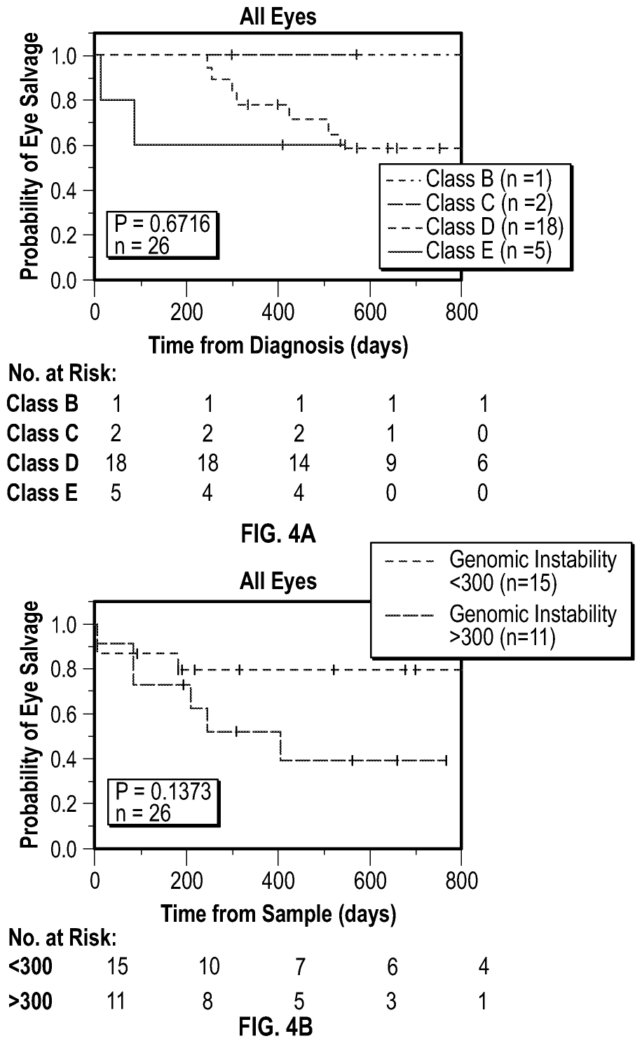

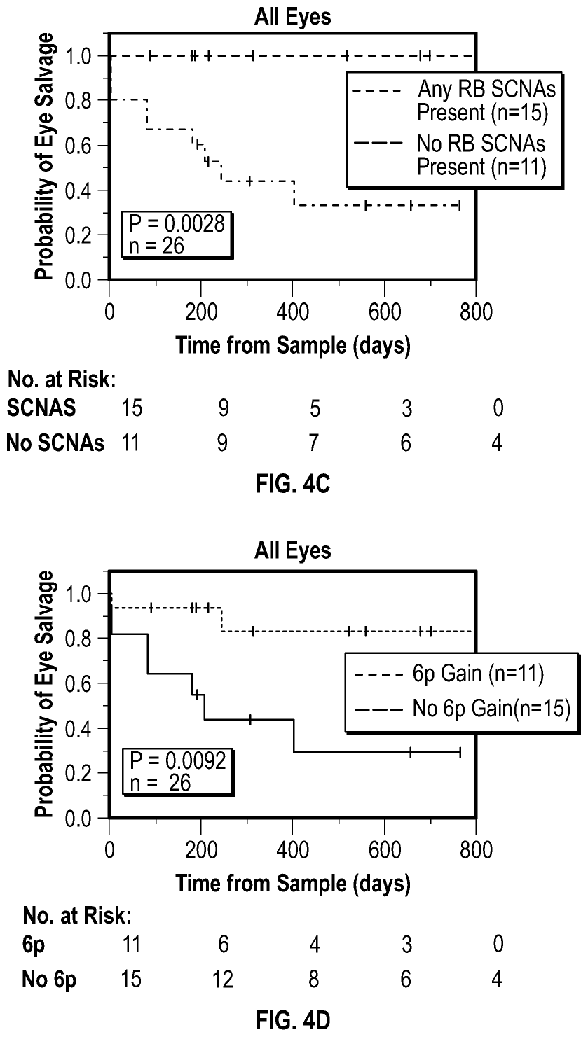

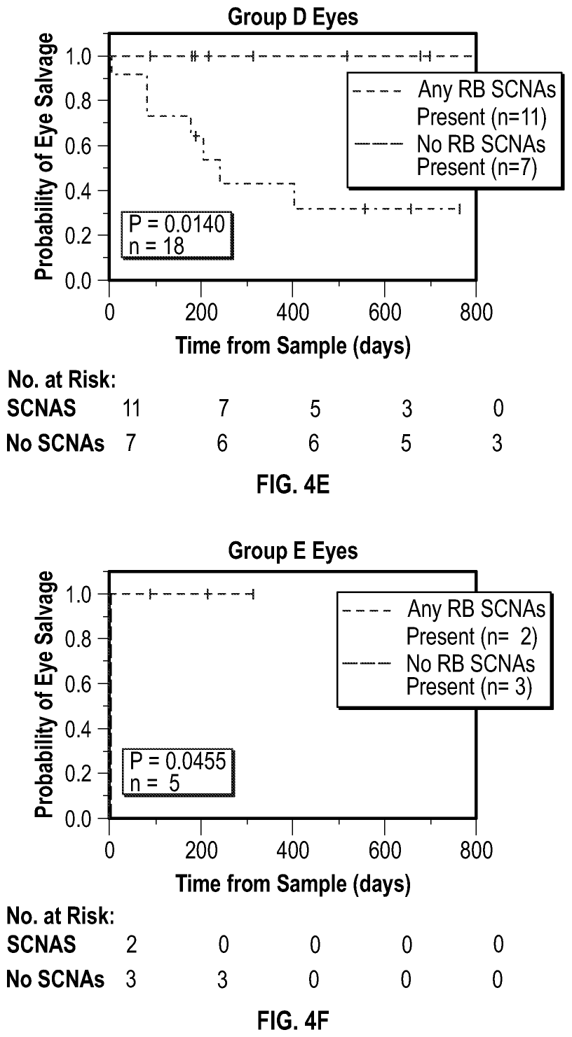

It is known that clinical classification (IIRC (3)) predicts eye salvage, however prognostic success remains limited for advanced eyes (4-7,35). Thus, to test whether genomic analysis of AH samples could better predict eye salvage, genomic instability, presence of RB SCNAs, and specifically 6p gain were analyzed in addition to clinical staging for ocular survival. Kaplan-Meier curves were evaluated at 1 standard deviation from the median follow-up (800 days). The primarily enucleated eyes (Cases 1,2,3) were removed as salvage was not attempted. FIG. 4A shows that IIRC classification stratifies eye survival by class, demonstrating that advanced Group D and E eyes have a lower likelihood of eye salvage than Group A. B or C eyes, although in this small data set this was not significant (p=0.6716).

Ocular survival was then evaluated based on AH genomics. Overall genomic instability was evaluated to determine if it was a useful biomarker for eye salvage with cox regression analysis, which was not shown to be predictive (p=0.5882, 95% CI:0.9982-1.0025). Then 300 was used as a marker of ‘high’ genomic instability which also did not predict ocular survival based on Kaplan-Meier analysis (FIG. 4B, log-rank, p=0.1373). However, the presence of RB SCNAs in the AH does predict eye salvage significantly better than clinical staging alone (FIG. 4C, p=0.0028). Within the RB SCNAs, gain of 6p in the AH was most predictive single SCNA of inability to salvage the eye (FIG. 4D, p=0.0092). Evaluation of both clinical and genomic information (the presence of RB SCNAs) in Group D and E eyes also demonstrates that use of genomic data significantly increased the predictive value of tumor relapse requiring enucleation in these advanced eyes (FIG. 4E p=0.0140; 4F p=0.0455). The odds of an eye requiring enucleation were 20 times greater if any RB SCNA was present in the AH (OR=20, 95% CI:2.1-195.0).

e. Longitudinal Evaluation of AH Samples May Predict Tumor Remission and Relapse.

Despite the relative genomic stability of the AH samples (FIG. 2) measurable differences were observed in the amplitude of these alterations over the course of treatment in a manner that correlated with clinical response to therapy. The changes in amplitude were interpreted to reflect the fraction of tumor DNA in the total AH cfDNA and by inference, tumor activity.

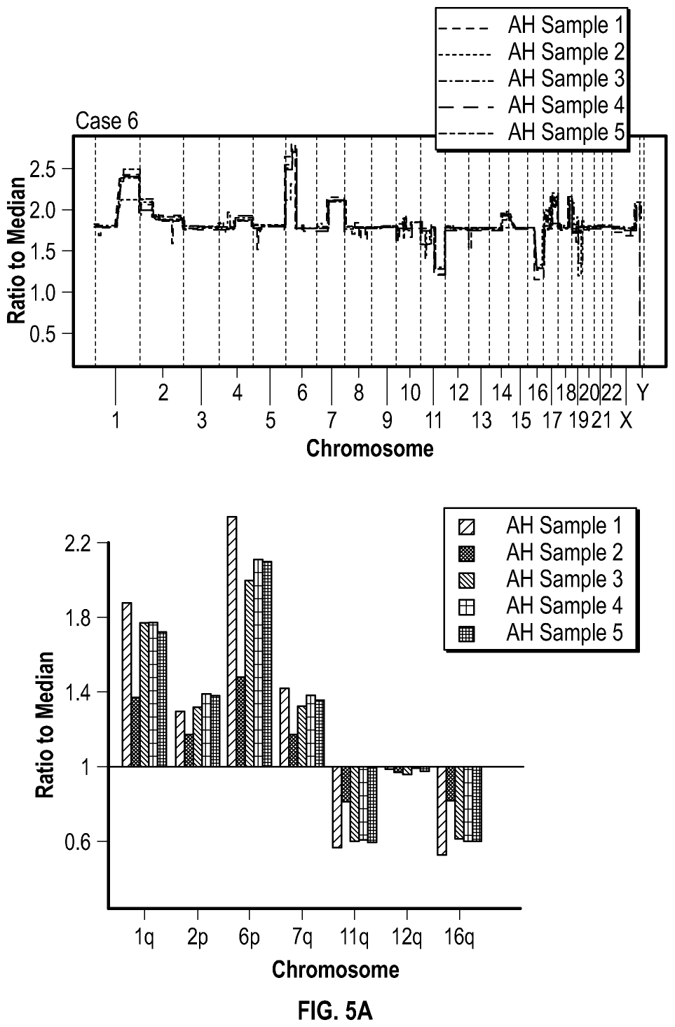

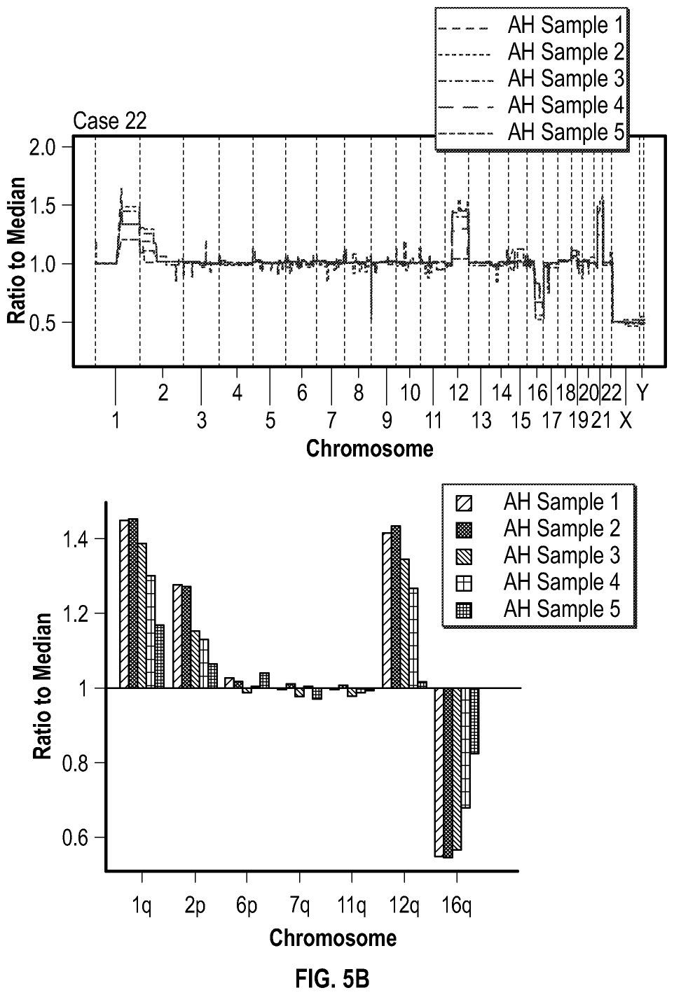

Two patients in this cohort had >4 samples of AH available for evaluation. Case 22 had 5 AH samples taken during intravitreal injection of chemotherapy in which the seeds were treated successfully, the main retinal tumor did not recur, and this eye was successfully salvaged. Comparison of the AH profiles over time showed a decreasing burden of tumor-derived DNA in the AH with no additional chromosomal alterations appearing. In fact, a decrease in the amplitude of alterations below threshold was seen in the last sample. Thus, correlating with decreased tumor-derived DNA and tumor response to therapy (FIG. 5B).

In contrast, Case 6 (previously described (21)), had 7 AH samples of which 5 had acceptable read alignment. Evaluation of the AH SCNA profiles demonstrated an initial decrease in the amplitude of alterations indicating a reduced amount of tumor DNA in the AH and a positive response to the intravitreal chemotherapy. However, the subsequent AH samples show increased amplitude, correlating clinically with active tumor recurrence; this eye subsequently required enucleation. The AH sample at the time of enucleation demonstrated increased number genomic events and instability (FIG. 5A).

Amplitude changes specifically in 6p also correlated with clinical outcomes. Two eyes in the salvaged group had small amplitude gain in 6p in the first AH sample only, becoming undetectable in later samples (Cases 21, 25) (Table 1). Similarly, one eye (Case 11) that required enucleation did not initially have a gain of 6p. However, with tumor relapse, 6p gain was present in subsequent AH samples.

Taken together, these results indicate that increase or decrease in SCNA amplitude can be useful as a real-time predictor of tumor response during treatment and demonstrates the utility of longitudinal evaluation of the AH during therapy.

### DISCUSSION

Herein is presented a novel analysis of retinoblastoma tumor-DNA in 63 separate AH samples, the majority of which were taken during active treatment. Evaluation of this larger data set provided further evidence that the AH ‘surrogate tumor biopsy’ (21) is a valid source of tumor-derived cfDNA and is representative of the genomic state of the tumor. With access to tumor DNA in vivo, differences were identified in the SCNA profiles from eyes that were salvaged and those that required enucleation. The differences in these genomic profiles significantly impacted the prediction of therapeutic tumor response. Notably, lack of a 6p gain confers a significant survival benefit for the eye (p=0.0092); stated conversely, the presence of 6p gain in the AH was associated with a 10 times increased risk of an eye requiring enucleation. It was further demonstrated that the overall amplitude of genomic alterations could provide a real-time measure of therapeutic response.

While the analysis of this larger sample set provided further support for the clinical utility of the AH, it should be noted that cfDNA taken from a cancer patient, whether from blood or AH, is a variable mixture of normal DNA and DNA shed from the tumor. Thus, measurements of copy number and peak amplitude of alterations reflect both the intrinsic genomic state of the tumor and also the overall quantity of tumor DNA in the fluid. These AH samples were not taken at diagnosis, but rather after initial chemotherapy at the time of adjuvant intravitreal injection of melphalan, or at the time of a tumor recurrence that required secondary enucleation. It was retrospectively observed that enucleated eyes had a higher frequency of RB SCNAs, with greater amplitude of alterations, than salvaged eyes. Thus, the AH SCNA profiles with minimal alterations that were seen in the salvaged eyes may reflect a tumor with similarly few copy number alterations, or rather the response of the tumor to previous chemotherapy and thus a low fraction of tumor-derived cfDNA in the AH, or both.

Of the RB SCNAs, 6p gain was the most frequently identified SCNA in the AH. Gains of 6p are also the most common genomic changes observed in retinoblastoma tumors (14,17). Driver genes for tumorigenesis associated with 6p gain have been postulated including DEK and E2F3 (10,14,36). DEK encodes a DNA-binding protein that acts as an oncogene in multiple cancers (37,38) and E2F3 is involved in transcriptional cell-cycle control, regulated by the retinoblastoma protein (pRB) (39). A more exact delineation of the mechanism of 6p gain in retinoblastoma tumorigenesis has been hindered by the fact that focal SCNAs outside of the 2p arm (MYCN) are rare (40), thus the minimum region of gain is not refined to the single gene level. Nonetheless herein, using 6p gain as a biomarker in the AH allowed, for the first time, correlation of the presence of this highly recurrent SCNA with clinical outcomes in retinoblastoma eyes undergoing treatment. It was found that both the frequency and the amplitude of 6p gain were significantly higher in the cohort of eyes that required enucleation and had significant predictive value.

Use of a biopsy to predict therapeutic response (41), risk of metastatic disease (42-44), and survival (45) for other malignancies, even other intraocular malignancies, has dramatically impacted the ability to provide precision medicine for cancer patients. Liquid biopsies based on circulating tumor cells and cfDNA in the blood or other fluids have been explored for other cancers as means to further prognosticate therapeutic outcomes without the need for invasive tissue biopsy (46-50).

### BIBLIOGRAPHY

1. Fabian I D, Stacey A W, Johnson K C, Chowdhury T, Duncan C, Reddy M A, et al. Primary enucleation for group D retinoblastoma in the era of systemic and targeted chemotherapy: the price of retaining an eye. Br J Ophthalmol 2017 doi 10.1136/bjophthalmol-2017-310624.
2. Mendoza P R, Grossniklaus H E. Therapeutic Options for Retinoblastoma. Cancer Control 2016; 23(2):99-109 doi 10.1177/107327481602300203.
3. Linn Murphree A. Intraocular retinoblastoma: the case for a new group classification. Ophthalmol Clin North Am 2005; 18(1):41-53, viii doi 10.1016/j.ohc.2004.11.003.
4. Shields C L, Mashayekhi A, Au A K, Czyz C, Leahey A, Meadows A T, et al. The International Classification of Retinoblastoma predicts chemoreduction success. Ophthalmology 2006; 113(12):2276-80 doi 10.1016/j.ophtha.2006.06.018.
5. Berry J L, Jubran R, Kim J W, Wong K, Bababeygy S R, Almarzouki H, et al. Long-term outcomes of Group D eyes in bilateral retinoblastoma patients treated with chemoreduction and low-dose IMRT salvage. Pediatr Blood Cancer 2013; 60(4):688-93 doi 10.1002/pbc.24303.
6. Kaliki S, Shields C L, Rojanaporn D, Al-Dahmash S, McLaughlin J P, Shields J A, et al. High-risk retinoblastoma based on international classification of retinoblastoma: analysis of 519 enucleated eyes. Ophthalmology 2013; 120(5):997-1003 doi 10.1016/j.ophtha.2012.10.044.
7. Shields C L, Manjandavida F P, Lally S E, Pieretti G, Arepalli S A, Caywood E H, et al. Intra-arterial chemotherapy for retinoblastoma in 70 eyes: outcomes based on the international classification of retinoblastoma. Ophthalmology 2014; 121(7):1453-60 doi 10.1016/j.ophtha.2014.01.026.
8. Mallipatna A, Gallie B L, Chevez-Barrios P, Lumbroso-Le Rouic L, Chantada G L, Doz F, et al. AJCC Cancer Staging Manual Vol. 8th Edition. M. B. Amin SBE, & F. L. Greene, editor: Springer; 2017.
9. Kooi I E, Mol B M, Massink M P, Ameziane N, Meijers-Heijboer H, Dommering C J, et al. Somatic genomic alterations in retinoblastoma beyond RB1 are rare and limited to copy number changes. Sci Rep 2016; 6:25264 doi 10.1038/srep25264.
10. Kooi I E, Mol B M, Massink M P, de Jong M C, de Graaf P, van der Valk P, et al. A Meta-Analysis of Retinoblastoma Copy Numbers Refines the List of Possible Driver Genes Involved in Tumor Progression. PLoS One 2016; 11(4):e0153323 doi 10.1371/journal.pone.0153323.
11. Li W L, Buckley J, Sanchez-Lara P A, Maglinte D T, Viduetsky L, Tatarinova T V, et al. A Rapid and Sensitive Next-Generation Sequencing Method to Detect RB1 Mutations Improves Care for Retinoblastoma Patients and Their Families. J Mol Diagn 2016; 18(4):480-93 doi 10.1016/j.jmoldx.2016.02.006.
12. Rushlow D E, Mol B M, Kennett J Y, Yee S, Pajovic S, Theriault B L, et al. Characterisation of retinoblastomas without RB1 mutations: genomic, gene expression, and clinical studies. Lancet Oncol 2013; 14(4):327-34 doi 10.1016/S1470-2045(13)70045-7.
13. Theriault B L, Dimaras H, Gallie B L, Corson T W. The genomic landscape of retinoblastoma: a review. Clin Exp Ophthalmol 2014; 42(1):33-52 doi 10.1111/ceo.12132.
14. Corson T W, Gallie B L. One hit, two hits, three hits, more? Genomic changes in the development of retinoblastoma. Genes Chromosomes Cancer 2007; 46(7):617-34 doi 10.1002/gcc.20457.
15. Dimaras H, Khetan V, Halliday W, Orlic M, Prigoda N L, Piovesan B, et al. Loss of RB1 induces non-proliferative retinoma: increasing genomic instability correlates with progression to retinoblastoma. Hum Mol Genet 2008; 17(10):1363-72 doi 10.1093/hmg/ddn024.
16. Kapatai G, Brundler M A, Jenkinson H, Kearns P, Parulekar M, Peet A C, et al. Gene expression profiling identifies different sub-types of retinoblastoma. Br J Cancer 2013; 109(2):512-25 doi 10.1038/bjc.2013.283.
17. Cano J, Oliveros O, Yunis E. Phenotype variants, malignancy, and additional copies of 6p in retinoblastoma. Cancer Genet Cytogenet 1994; 76(2):112-5.
18. Grasemann C, Gratias S, Stephan H, Schuler A, Schramm A, Klein-Hitpass L, et al. Gains and overexpression identify DEK and E2F3 as targets of chromosome 6p gains in retinoblastoma. Oncogene 2005; 24(42):6441-9 doi 10.1038/sj.onc.1208792.
19. Karcioglu Z A. Fine needle aspiration biopsy (FNAB) for retinoblastoma. Retina 2002; 22(6):707-10.
20. Karcioglu Z A, Gordon R A, Karcioglu G L. Tumor seeding in ocular fine needle aspiration biopsy. Ophthalmology 1985; 92(12):1763-7.
21. Berry J L, Xu L, Murphree A L, Krishnan S, Stachelek K, Zolfaghari E, et al. Potential of Aqueous Humor as a Surrogate Tumor Biopsy for Retinoblastoma. JAMA Ophthalmol 2017 doi 10.1001/jamaophthalmol.2017.4097.
22. Francis J H, Abramson D H, Ji X, Shields C L, Teixeira L F, Schefler A C, et al. Risk of Extraocular Extension in Eyes With Retinoblastoma Receiving Intravitreous Chemotherapy. JAMA Ophthalmol 2017; 135(12):1426-9 doi 10.1001/jamaophthalmol.2017.4600.
23. Munier F L, Soliman S, Moulin A P, Gaillard M C, Balmer A, Beck-Popovic M. Profiling safety of intravitreal injections for retinoblastoma using an anti-reflux procedure and sterilisation of the needle track. Br J Ophthalmol 2012; 96(8):1084-7 doi 10.1136/bjophthalmol-2011-301016.
24. Baslan T, Kendall J, Rodgers L, Cox H, Riggs M, Stepansky A, et al. Genome-wide copy number analysis of single cells. Nat Protoc 2012; 7(6):1024-41 doi 10.1038/nprot.2012.039.
25. Baslan T, Kendall J, Rodgers L, Cox H, Riggs M, Stepansky A, et al. Corrigendum: Genome-wide copy number analysis of single cells. Nat Protoc 2016; 11(3):616 doi 10.1038/nprot0316.616b.
26. 2017 UCSC Genome Browser. <https://genome.ucsc.edu/>.
27. Langmead B, Salzberg S L. Fast gapped-read alignment with Bowtie 2. Nat Methods 2012; 9(4):357-9 doi 10.1038/nmeth.1923.
28. Langmead B, Trapnell C, Pop M, Salzberg S L. Ultrafast and memory-efficient alignment of short DNA sequences to the human genome. Genome Biol 2009; 10(3):R25 doi 10.1186/gb-2009-10-3-r25.
29. Li H. A statistical framework for SNP calling, mutation discovery, association mapping and population genetical parameter estimation from sequencing data. Bioinformatics 2011; 27(21):2987-93 doi 10.1093/bioinformatics/btr509.
30. Huber W, Carey V J, Gentleman R, Anders S, Carlson M, Carvalho B S, et al. Orchestrating high-throughput genomic analysis with Bioconductor. Nat Methods 2015; 12(2):115-21 doi 10.1038/nmeth.3252.
31. Varin T, Bureau R, Mueller C, Willett P. Clustering files of chemical structures using the Szekely-Rizzo generalization of Ward's method. J Mol Graph Model 2009; 28(2):187-95 doi 10.1016/j.jmgm.2009.06.006.
32. Strauss T, von Maltitz M J. Generalising Ward's Method for Use with Manhattan Distances. PLoS One 2017; 12(1):e0168288 doi 10.1371/joumal.pone.0168288.
33. Navin N, Hicks J. Future medical applications of single-cell sequencing in cancer. Genome Med 2011; 3(5):31 doi 10.1186/gm247.
34. Kooi I E, Mol B M, Moll A C, van der Valk P, de Jong M C, de Graaf P, et al. Loss of photoreceptomess and gain of genomic alterations in retinoblastoma reveal tumor progression. EBioMedicine 2015; 2(7):660-70 doi 10.1016/j.ebiom.2015.06.022.
35. Berry J L, Shah S, Bechtold M, Zolfaghari E, Jubran R, Kim J W. Long-term outcomes of Group D retinoblastoma eyes during the intravitreal melphalan era. Pediatr Blood Cancer 2017; 64(12) doi 10.1002/pbc.26696.
36. Bowles E, Corson T W, Bayani J, Squire J A, Wong N, Lai P B, et al. Profiling genomic copy number changes in retinoblastoma beyond loss of RB1. Genes Chromosomes Cancer 2007; 46(2):118-29 doi 10.1002/gcc.20383.
37. von Lindem M, Fornerod M, Soekarman N, van Baal S, Jaegle M, Hagemeijer A, et al. Translocation t(6;9) in acute non-lymphocytic leukaemia results in the formation of a DEK-CAN fusion gene. Baillieres Clin Haematol 1992; 5(4):857-79.
38. Carro M S, Spiga F M, Quarto M, Di Ninni V, Volorio S, Alcalay M, et al. DEK Expression is controlled by E2F and deregulated in diverse tumor types. Cell Cycle 2006; 5(11):1202-7 doi 10.4161/cc.5.11.2801.
39. Adams M R, Sears R, Nuckolls F, Leone G, Nevins J R. Complex transcriptional regulatory mechanisms control expression of the E2F3 locus. Mol Cell Biol 2000; 20(10):3633-9.
40. Mol B M, Massink M P, van der Hout A H, Dommering C J, Zaman J M, Bosscha M I, et al. High resolution SNP array profiling identifies variability in retinoblastoma genome stability. Genes Chromosomes Cancer 2014; 53(1):1-14 doi 10.1002/gcc.22111.
41. Yun M, Bai H Y, Zhang J X, Rong J, Weng H W, Zheng Z S, et al. ULKI: a promising biomarker in predicting poor prognosis and therapeutic response in human nasopharygeal carcinoma. PLoS One 2015; 10(2):e0117375 doi 10.1371/joumal.pone.0117375.
42. Onken M D, Worley L A, Ehlers J P, Harbour J W. Gene expression profiling in uveal melanoma reveals two molecular classes and predicts metastatic death. Cancer Res 2004; 64(20):7205-9 doi 10.1158/0008-5472.CAN-04-1750.
43. Onken M D, Worley L A, Tuscan M D, Harbour J W. An accurate, clinically feasible multi-gene expression assay for predicting metastasis in uveal melanoma. J Mol Diagn 2010; 12(4):461-8 doi 10.2353/jmoldx.2010.090220.
44. Harbour J W. A prognostic test to predict the risk of metastasis in uveal melanoma based on a 15-gene expression profile. Methods Mol Biol 2014; 1102:427-40 doi 10.1007/978-1-62703-727-3_22.
45. Hicks J, Krasnitz A, Lakshmi B, Navin N E, Riggs M, Leibu E, et al. Novel patterns of genome rearrangement and their association with survival in breast cancer. Genome Res 2006; 16(12):1465-79 doi 10.1101/gr.5460106.
46. Tarazona N, Cervantes A. Liquid biopsy: another tool towards tailored therapy in colorectal cancer. Ann Oncol 2017 doi 10.1093/annonc/mdx641.
47. Shukuya T, Patel S, Shane-Carson K, He K, Bertino E M, Shilo K, et al. Lung Cancer Patients with Germline Mutations detected by next generation sequencing and/or liquid biopsy. J Thorac Oncol 2017 doi 10.1016/j.jtho.2017.09.1962.
48. Zhang W, Xia W, Lv Z, Ni C, Xin Y, Yang L. Liquid Biopsy for Cancer: Circulating Tumor Cells, Circulating Free DNA or Exosomes? Cell Physiol Biochem 2017; 41(2):755-68 doi 10.1159/000458736.
49. von Bubnoff N. Liquid Biopsy: Approaches to Dynamic Genotyping in Cancer. Oncol Res Treat 2017; 40(7-8):409-16 doi 10.1159/000478864.
50. Baslan T, Kendall J, Ward B, Cox H, Leotta A, Rodgers L, et al. Optimizing sparse sequencing of single cells for highly multiplex copy number profiling. Genome Res 2015; 25(5):714-24 doi 10.1101/gr.188060.114.

### Example 2

Testing the aqueous humor for markers of disease is superior to testing the blood. 18 matched aqueous humor (AH) and blood samples were tested for the presence of tumor derived DNA and chromosomal alterations (e.g. somatic copy number alterations, SCNA) in order to demonstrate that testing the AH is superior to testing the blood. Five samples of AH taken at diagnosis and 13 samples at the time of intravitreal injection of chemotherapy were compared to matched blood samples from 16 patients (2 patients had both eyes included for 18 AH samples). The presence of any detectable SCNA in the AH was 14/18 and 0/16 in the blood. The median concentration of cfDNA in blood is 5.3 ng/ml (std dev 41.5) however there was no indication that tumor-derived cfDNA was present in the blood and no SCNAs present for evaluation in the blood.

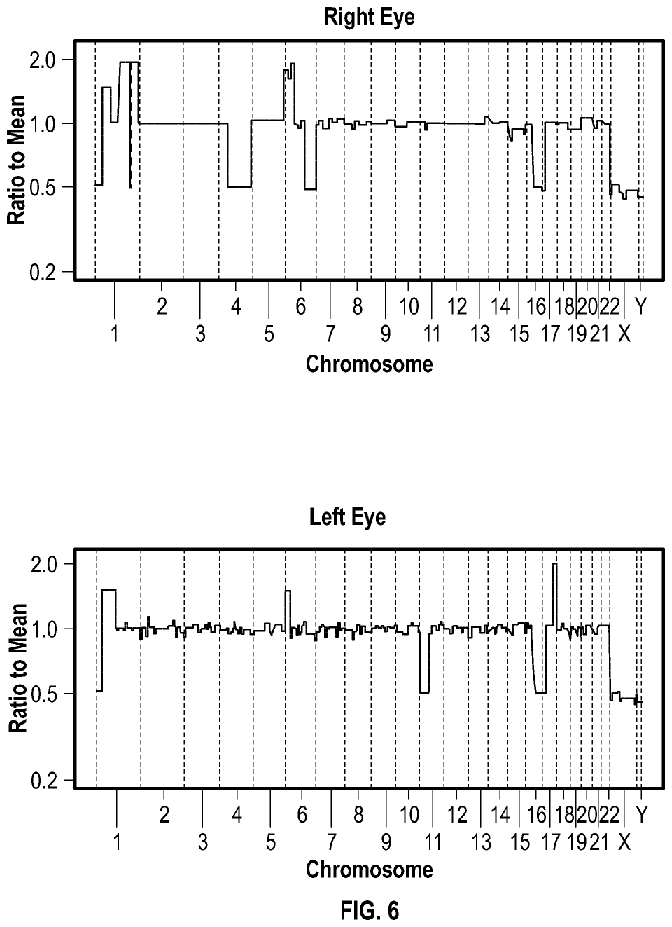

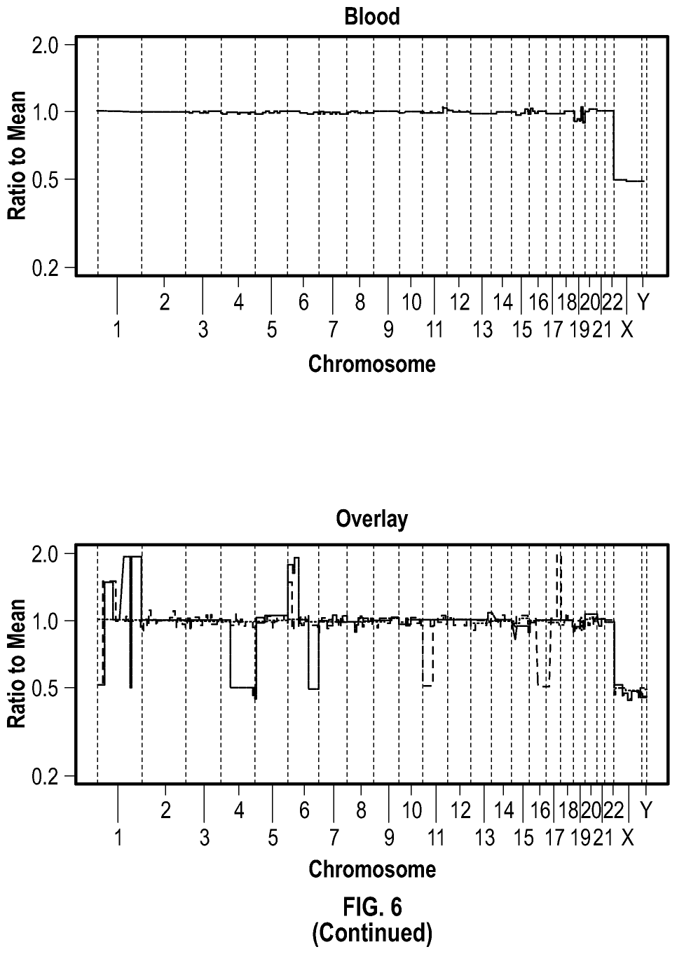

FIG. 6 demonstrates representative genomic profiles from the AH and the blood. Patient 1 has tumor in both eyes with different copy number profiles demonstrating, as shown previously, that tumors in different eyes develop different chromosomal alterations with separate prognostic implications (right eye BLUE, left eye GREEN). There was no detectable tumor-derived cell-free DNA in the blood from this child (RED).

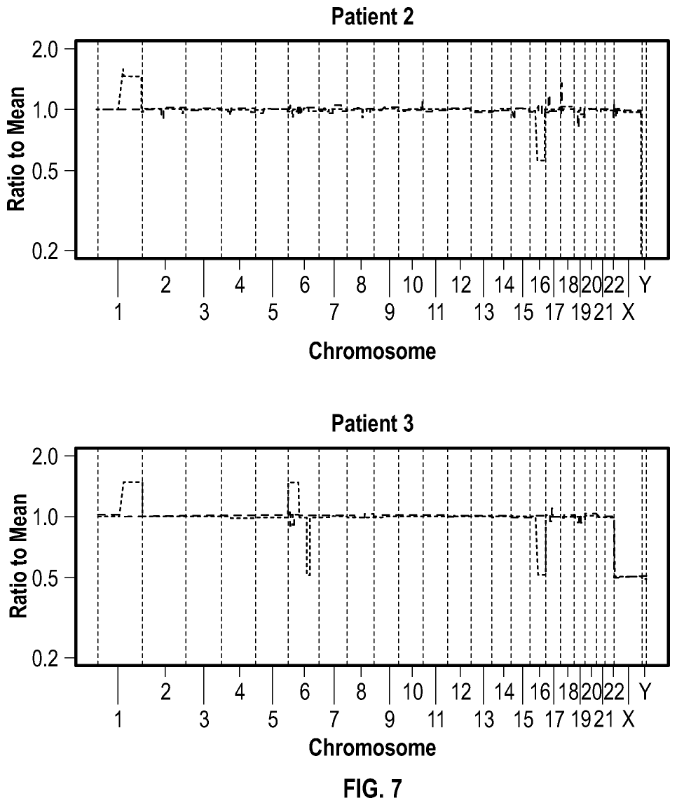

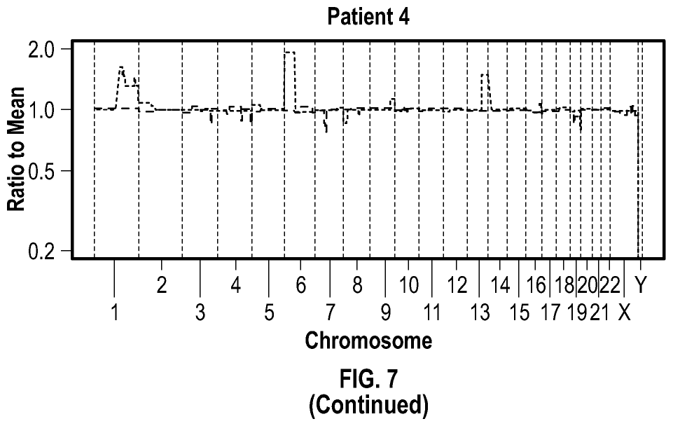

Similarly (FIG. 7), patients 2, 3, 4 have a flat profile in the blood without detectable SCNAs in the cfDNA in the blood (RED), however there are clear chromosomal changes found in the aqueous humor (BLUE).

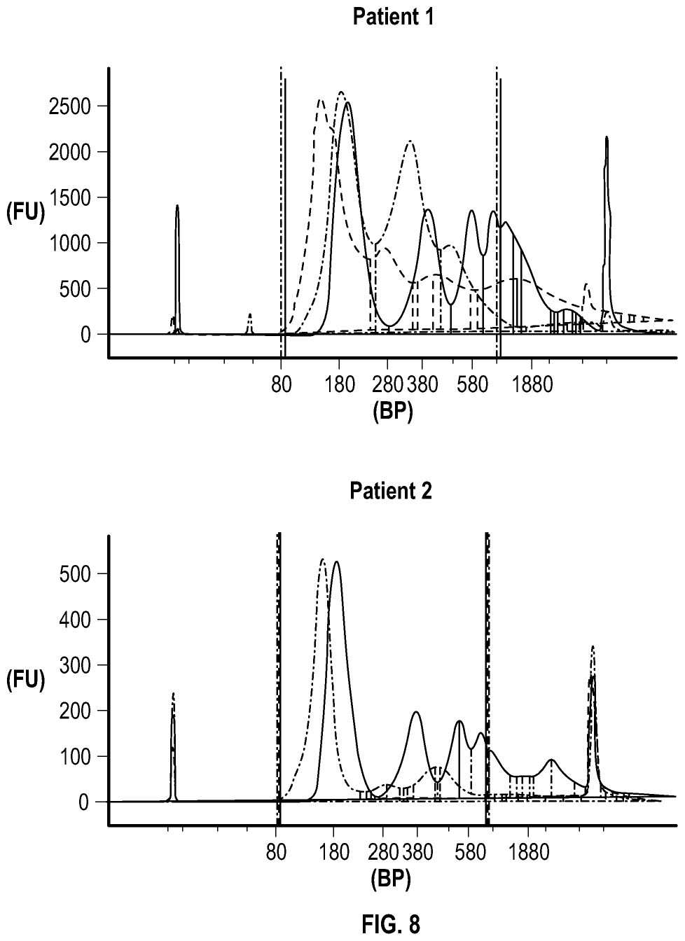

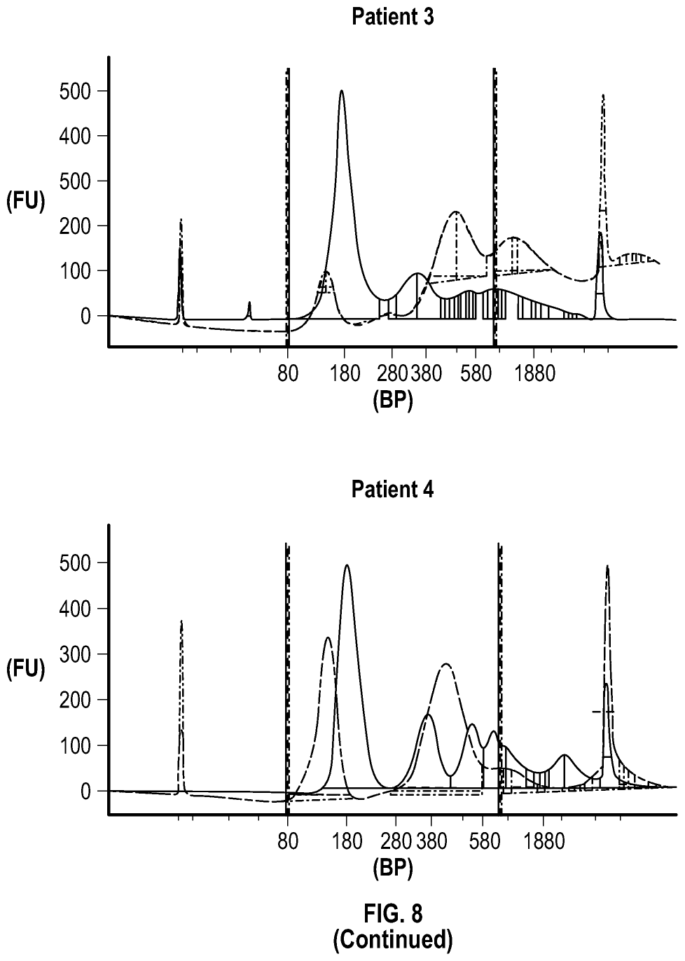

The DNA size distribution of cfDNA in the AH peaked at 157 basepairs (bp) versus 184 bp in the blood. This is consistent with other studies that have demonstrated that tumor derived cell-free DNA is in fact shorter than circulating cell-free DNA. This further supports that tumor DNA is found in the AH and not in the blood for these patients. FIG. 8 shows the peak cell-free DNA fragment size in the AH (blue, green second eye) vs the blood (red).

### BIBLIOGRAPHY

Mouliere F, Piskorz A M, Chandrananda D, et al, 2017. Selecting short DNA fragments in plasma improves detection of circulating tumour DNA. Available: http://biorxiv. org/lookup/doi/
Jiang P, Lo Y M D. The long and short of circulating cell-free DNA and the ins and outs of molecular diagnostics. Trends Genet 2016; 32:360-71.
Snyder et al., Cell-free SNA compromises an In Vivo Nucleosome Footprint that Informs Its Tissues-of-Origin. Cell 2016, 164, 57-68.

### Example 3

Even in eyes with small tumor volumes (e.g., less advanced disease) tumor derived DNA can be found in the AH thus the prognostic testing done in more advanced eyes can also be tested and applied to less advanced eyes. To date diagnostic testing has been carried out on 5 eyes, 2 of which were ‘less advanced’ eyes and both showed evidence of tumor derived cell-free DNA, RNA and miRNA. Diagnostic AH in two eyes of the same patient with bilateral retinoblastoma, with matched blood, is shown in FIG. 6.

### Example 4

Retinoblastoma is a genetic tumor caused by two mutations in the RB1 tumor suppressor gene, in ⅓ of patients one of the mutations is present in all cells of the body (called a germline mutation) and thus present in the blood, however in ⅔ of the patients the mutations are only in the tumor (called somatic mutations). Finally, in either type of patient to find both mutations tumor DNA needs to be present, which previously was only available from tumor tissue in enucleated eyes. Because tumor DNA is present in the AH we can now assay both RB1 mutations in the AH. As disclosed herein, pathogenic MYCN amplifications can also be captured (2% of unilateral cases have primary MYCN amplification, many tumors have secondary MYCN-amplification).

| Sample ID | RB1 mutation_1 | RB1 mutation_2 |
|---|---|---|
| 2016_012_ES_AH | p.R251* | p.R320* |
| 2016_012_ES_tumor | p.R251* | p.R320* |
| 2016_021_ES_AH | p.L809* | MYCN-Amp |

### BIBLIOGRAPHY

Gerrish A, Stone E. Clokie S, et a. Non-invasive diagnosis of retinoblastoma using cell-free DNA from aqueous humour British Journal of Ophthalmology Published Online First: 11 Feb. 2019. doi: 10.1136/bjophthalmol-2018-313005

### Example 5

Epigenetic mechanisms of prognosis for retinoblastoma: miRNA in tumor has been shown to have prognostic value as a biomarker, it can be harnessed in the AH and it shows prognostic implications as well from AH testing.

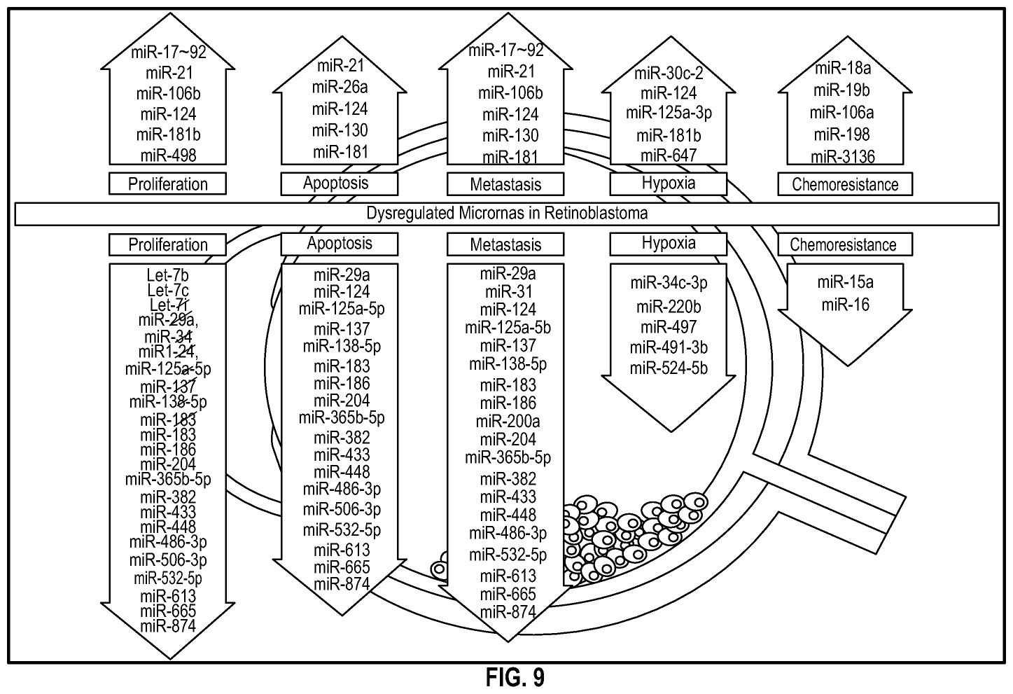

There are multiple studies on miRNA in retinoblastoma tumor. FIG. 9 provides a graphic, as well as Table 2, below, summarizing data.

TABLE 1
microRNAs that are differentially expressed in human retinoblastoma.

| microRNAs | Reference/Methods |
|---|---|
| let-7e; miR-513; miR-518c; miR-129; miR-198; miR-320; | Zhao et al., 2009/A |
| miR-373; miR-492; miR-494; miR-498; miR-503 | |
| let7a; let-7f; miR-R2; miR-7; miR-9; miR-16; miR-17a; miR-9; | Conkrite et al., 2011/A |
| miR-17a, miR-20a; miR-25; miR-26a; miR-30b; miR-30d; | |
| miR-92a; miR-93a; miR-96; miR-99b; miR-101; miR-103; | |
| miR-106b; miR-124; miR-143; miR-148b; miR-181a; miR-183; | |
| miR-216a; miR-217; miR-378; miR-1246 | |
| let7a; let-7b; let-7c; miR-10a; miR-10b; miR-20a; miR-21; | Li et al., 2012/-A |
| miR-28; miR-29b, miR-30a-3p; miR-30b; miR-30c; miR-30d; | |
| miR-99a; miR-99b; miR-100; miR-103; miR-107; miR-124a; | |
| miR-125a; miR-125b; miR-133a; miR-136 miR-141; miR-145; | |
| miR-146a; miR-155; miR-181a; miR-181b; miR-182; miR-183; | |
| miR-190; miR-191; miR-206; miR-210; miR-222; miR-301; | |
| miR-302a; miR-302b; miR-320; miR330; miR-335; miR-342; | |
| miR-368; miR-373; miR-380-5p; miR-382; miR-423; miR-433; | |
| miR-451; miR-452; miR-491 | |
| miR-378-34a | Dalgard et al., 2009/B; C |
| let-7c; let-7i; let-7g; miR-10a; miR-10b; miR-28-5p; miR-29a; | Jo et al., 2011/A |
| miR-29b; miR-29c; miR-34a; miR-34b; miR-34c-5p; miR-96; | |
| miR-99a; miR-100; miR-124; miR-125b; miR-130a; miR-132; | |
| miR-135b; miR-137; miR-142-3p; miR-142-5p; miR-149; miR- | |
| 181a; miR-182; miR-183; miR-193a-3p; miR-193b; miR-199a- | |
| 3p; miR-214; miR-224; miR-338-3p; miR-363; miR-374a; | |
| miR-375; miR-376a; miR-505 | |

A. Microarray Analysis; B. Semiquantitative RF PCR; C. Real-Time pPCR

More specifically:

### N-myc:

Let-7 and miR-34 family members both target the N-myc oncogene and have been postulated to reduce N-Myc's effect as an oncogene in retinoblastoma (1-3). An enantiomer of alkannin, Shikonin, was shown to inhibit proliferation of RB cells through upregulating both miR-34a and miR-202, which directly targets the N-myc oncogene for degradation (4). MYCN expression is known to upregulate the Mir-17-92 family in other cancers (5-8). An in vivo MYCN-driven retinoblastoma tumor initially responded to MYCN suppression with suppression of tumor proliferation and increased cell death (9). Wu et al. reported complete elimination of retinoblastoma tumor from the anterior chamber in 66 of 71 eyes (21+/−13 days). Removal of MYCN did not sustain suppression though as 56 of the 66 eyes saw a return of highly proliferative tumor in the anterior chamber of the eye (87+/−51 days). A subset, but not most, of the returning tumors showed genomic amplification of the MYCN target gene Mir-17-92 family. Mir-17-92 can play a role in some MYCN-independent tumor reemergence, but Mir-17-92 overexpression was not shown to rescue MYCN repression in vitro.

### Dicer1

Monoallelic loss of Dicer1 promotes retinoblastoma, while homozygous loss inhibits tumorigenesis (1). Let-7 and miR-34 share N-myc as a target and are upregulated in normal retina. In the heterozygous Dicer1 retina both Let-7 and miR-34 were downregulated.

### VEGFA:

miR-497 negatively regulates VEGFA to inhibit cell proliferation, migration and invasion in retinoblastoma in vitro (10).

### DRAM2:

DRAM2 (DNA-damage-regulated autophagy modulator protein 2) induces the autophagy process and is an effector molecule for p53-mediated apoptosis (11). miR-125B directly targets DRAM2 which significantly suppressed retinoblastoma cell apoptosis in vitro (12).

### STX17

Metastasis associated lung adenocarcinoma transcript 1 (MALAT1) promotes retinoblastoma cell autophagy via inhibiting miR-124 downregulation of Syntaxin 17, a Soluble NSF Attachment Protein receptor (SNARE) that mediates autophagosome formation and fusion with the lysosome membrane (13, 14).

### Hypoxia Inducible Factor-1Alpha

miR-320 upregulates autophagy in retinoblastoma cells by upregulating a downstream target hypoxia inducible factor 1alpha (15).

### STAT3

miR-124 suppresses retinoblastoma cell proliferation, migration and invasion and induced cell apoptosis in vivo in part by targeting signal transducer and activator of transcription 3 (STAT3) (16). miR-29a inhibits tumorigenesis by downregulating STAT3 expression in retinoblastoma cells (17). Jo et al. reported a positive feedback loop between STAT3/miR-17-92 amendable to targeted siRNA (18). Treatment with miR inhibitors such as miR-18-5p, miR-19a-3p and mirR-19b-3p reduced expression levels of target genes of STAT3 like BCL2, BCL2L1, BIRC5 and MMP9.

### FASN and STK

miR-486-3p and miR-532-5p share reported retinoblastoma oncogene targets spleen tyrosine kinase (STK) and fatty acid synthase (FASN) (19, 20). In vitro overexpression of miR-486-3p and miR-532-5p increased apoptosis to about 30.9% and 30.6% compared to untransfected cells (21).

### Notch1

miR-433 inhibits retinoblastoma cell proliferation and metastasis in part by downregulating Notch1 expression (22).

### PAX6

miR-433 inhibits retinoblastoma cell proliferation and metastasis in part by downregulating PAX6 expression (22). miR-655 is normally downregulated in retinoblastoma cells (23) miR-655 is anti-tumorigenic by targeting PAX6. miR-655 regulating PAX6 reduces activity of the extracellular signal-regulated kinase and p38 mitogen-activated protein kinase signaling pathways in retinoblastoma cells. Increased expression of miR-365b-3p in retinoblastoma cells inhibits retinoblastoma cell proliferation by targeting PAX6, which lead to increased Gi cell phase arrest and cell apoptosis (24). Interestingly, there was a corresponding up-regulation of P21 and P27 with down-regulation of cdc2 and Cyclin D1 protein. miRNA-758 inhibits retinoblastoma tumorigenesis by targeting PAX6 which inactivated the PI3K/Akt pathway (25).

p53

p14ARF protein activates p53 by inhibiting MDM2 (26-30). miR-24 directly targets p14ARF mRNA to prevent the activation of p53 in retinoblastoma cells (31). Cooperative co-silencing of miR-17/20a and p53 decreased the viability of human retinoblastoma cells with a nonexistent effect on retinogenesis (32). Differential miRNA profile in SNUOT-Rb1 cells and Y79 Cells

| | | | | | |
|---|---|---|---|---|---|
| miR-199a-3p, miR-99a, miR-125b, miR-214, | upregulated | microarray | SNUOT-Rb1 cell | NA | 103 |
| miR-10b, miR-29b, miR-100, miR-224, miR- | | | line | | |
| 505, miR-29a, miR-363, miR-10a, miR-137, let-7c, | | | | | |
| miR-193a-3p, miR-374a, miR-130a, miR-29c, | | | | | |
| miR-335, miR-181a, miR-28-5p, miR-376a | | | | | |
| miR-124, miR-142-3p, miR-34a, miR-135b, | upregulated | microarray | Y79 cell line | NA | 103 |
| miR-96, miR-142-5p, miR-183, miR-338-3p, miR- | | | | | |
| 193b, let-7i, miR-182, miR-149, miR-let-7g, | | | | | |
| miR-34c-5p, miR-132, miR-34b | | | | | |

These differentially expressed miRNAs between the SNUOT-Rb1 and Y79 cell line are related with biological functions to progress retinoblastoma formation such as cell cycle, cell death and cell division (33).

### MDM4

miR-191 binds to MDM4 mRNA and has decreased levels in retinoblastoma (34). Alternative transcripts of MDM4 mRNA in a primary retinoblastoma cohort (38/44) also had at least one allele insensitive to miR-191 regulation.

alpha-Enolase 1 (ENO1)

miR-22-3p prevents retinoblastoma cell proliferation through reducing the expression of alpha-enolase 1 (ENO1) (35).

### Erythroblastic Leukemia Viral Oncogene Homolog 3 (Erbb3)

Curcumin, a natural polyphenolic compound, upregulates the tumor-suppressor miRNA-22 (36, 37). miRNA-22 targeting the erythroblastic leukemia viral oncogene homolog 3 (Erbb3) inhibits cell proliferation and reduces migration in transfected miR-22 retinoblastoma cells.

### B7-H1

B7-H1 mRNA, which codes for a protein that impairs tumor immune surveillance, is a direct target of miR-513A-5p immunosuppression (38). Wu et al. reported the anticancer chemotherapy etoposide upregulates B7-H1 expression which might contribute to retinoblastoma chemoresistance (39).

### Beclin 1

Beclin 1 protein regulates tumor onset and progression through pro-autophagy. Mir-26A targets Beclin 1 mRNA (40).

### Caspase-3

Arsenic trioxide downregulates expression of miR-376a to mediate caspase-3 apoptosis (41). Caspase-3 was shown to be the target of miR-376a.

### E2F

E2F transcription factors induce miRNA-449A and -449b transcription that then target the expression of the E2F transcription factors, forming a feedback loop (42, 43). Interestingly Martin et al. reported both miR-449a and -449b were upregulated in their retinoblastoma tumor cohort (44). They proposed the inhibitory effects of both miRNAs are only significant at higher levels made attainable by transfection.

### E2F5

miR-613 downregulates E2F5 in retinoblastoma cells (45). HMGA1/HMGA2

In Mu et al.'s entire cohort of 28 nontumor retina samples let-7 has been reported to be robustly expressed, while reduced expression levels of let-7 appeared in 17 (39%) of retinoblastoma tumors (46). There is a significant inverse association between let-7 and high mobility group A2 while possible significance exists between let-7 and high mobility group A1. Downregulation of let-7 may have some effect on overexpression of HMGA1 and HMGA2 in the pathogenesis retinoblastoma. HMGA2 silencing in retinoblastoma cells has been observed to reduce cell proliferation in cultured RB cells and downregulate expression of oncogenic miRNA family's miR-17-92 and miR-106b-25 (47, 48).

### CDC25A

Huang et al. reported downregulation in let-7b on average 50-fold lower abundance comparing 9/10 retinoblastoma samples from different individuals than the average let-7b expression in five retina samples from healthy individuals (49). The under-expression of let-7b upregulates CDC25A expression in retinoblastoma.

### Cyclin D2

CyclinD2 is upregulated in retinoblastoma tissue and cell lines and has convincing evidence for maintaining an inverse relationship with levels of miR-204 in retinoblastoma (50).

### MMP-9

MMP-9 is upregulated in retinoblastoma tissue and cell lines and has convincing evidence for maintain an inverse relationship with levels of miR-204 in retinoblastoma (50). Wang et al. proposed the differentiation antagonizing non-protein coding RNA (DANCR) blocks targeting of MMP9 by miR-613 and miR-34c by binding and harboring both microRNAs (51).

### TAZ-EGFR

miR125a-5p targets the transcriptional co-activator with PDZ binding motif (TAZ) downregulating the epithelial growth factor receptor pathway and its downstream cell cycle components Cyclin E and CDK2 (52).

### ABCG2

miR-3163 targets ATP-binding cassette, subfamily G, member 2 (ABCG2) to induce apoptosis and anti-tumorigenesis in retinoblastoma cancer stem cells and inhibits multidrug resistance normally provided by pumping chemotherapy drugs out of cells (53).

### Epithelial Mesenchymal Transition

MiR-200c inhibits retinoblastoma cell migration by reverse epithelial mesenchymal transition (54). miR-613 inhibits tyrosine protein kinase Met (c-Met) to downregulate the epithelial mesenchymal transition in retinoblastoma cells. The LncRNA HOTAIR (HOX transcript antisense RNA) was found to be negatively regulate miR-613 (55).

### BAD and AKT

miR-21 targets BAD (Phospho-Ser155) and AKT (Phospho-Ser473) to inhibit apoptosis and promote tumorigenesis in retinoblastoma cells (56).

### PDCD4

miR-21 targets PDCD4 to downregulate Rb1 and subsequently suppress tumor formation (57).

### PTEN/PI3K/AKT

miR-21 inhibitor was shown to upregulate apoptosis by modulating levels of PDCD4, Bax and Bcl-2, inhibit cell migration and invasion by downregulating levels of MMP2 and MMP9 and miR-21 inhibits the PTEN/PI3K/AKT signaling pathway (58). miRNA-382 inhibits RB proliferation and invasion by downregulating the BDNF-mediated PI3K/AKT signaling pathway (59). miRNA-198 targets PTEN and upregulates the PI3K/AKT signaling pathway to promote cell proliferation and invasion in retinoblastoma (60).

### ROCK1

miRNA-448 targets ROCK1 to inhibit the PI3K/AKT signaling pathway and decreases cell proliferation and invasion and increases cellular apoptosis in retinoblastoma (61).

### PDCD10 and GATA6

miR-181b stimulates angiogenesis of retinoblastoma tumor in part by inhibiting PDCD10 and GATA6 (62).

### Epithelial Cell Adhesion Molecule

Epithelial cell adhesion molecule (EpCAM) promotes retinoblastoma tumorigenesis microRNAs Mir-130b and mir-181c that increase cell proliferation (63). EpCam downregulation resulted in significant decrease in the expression of miR-17-92 family suggesting high levels of EpCam in retinoblastoma promote miR-17-92 family expression (64).

### EZH2 and HDAC9

miR-101-3p targets enhancer of zeste homolog 2 (EZH2) and histone deacetylase (HDAC2) to inhibit cell proliferation of retinoblastoma cells (65).

### EZH2

Overexpression of EZH2 upregulates cell proliferation, colony formation and enhances cell migration and invasion (66, 67). miR-101 targets EZH2 to inhibit retinoblastoma cell proliferation and growth (68).

miR-34a

miR-34a is a product of p53 activation and miR-34a transfection of retinoblastoma cells downregulated levels of CCND1, CNNE2, CDK4, E2F3, EMP1, MDMX and SIRT1 (69).

### HMGB1

miR-34A targets high mobility group box 1 (HMGB1) to inhibit autophagy and improve chemotherapy-induced apoptosis in retinoblastoma cells (70) LRP6

miR-183 targets wnt co-receptor low-density lipoprotein receptor-related protein 6 (LRP6) to prevent cell proliferation and migration and invasion of retinoblastoma cells (71).

p21/Cip1/CDK/p130 axis

Upregulation of miR-17-92 family miRNA was insufficient to promote tumorigenesis but combined with inactivation of Rb/p107 lead to dramatic tumorigenesis (72). Conkrite et al. proposed a synergistic suppression of a p21/Cip1/CDK/p130 axis by miR-12-92 and Rb loss. miR-17/20 of the miR-17-92 family promote retinoblastoma cell proliferation.

### Pri-miRNA-17-92 Aptamer

An RNA aptamer can effectively target the primary-miRNA-17-92 and replace the mix of five antagomirs to prevent the maturation of miRNA-17-92 miRNAs (73).

### Long Non-Coding RNA H19

Long non-coding RNA H19 downregulates retinoblastoma tumorigenesis through binding and counteracting the miR-17-92 family (74).

Serum miR-17-92 in Retinoblastoma Diagnosis

miR-17-3P, miR-17-5P, miR-18a and miR-20a are significantly expressed in the serum of children with retinoblastoma (75). A micro fluidic mixer can detect significant differences of miR-18a in the serum of children with retinoblastoma Group E patients and same-age non-cancerous patients (76).

miRNA-143

miRNA-143 upregulates Bax, decreases Bcl-2 with apoptotic effects of retinoblastoma cells (77).

### SD-208

TGF-Beta-RI Kinase Inhibitor, SD-208, upregulates miRNAs 18a, 22a and 34a while downregulating miRNA 20a (78).

### Specificity Protein 1

miR-320 targeting specificity protein 1 reduced proliferation, migration and invasion of RB cells (79)

### Adam19

miR-145 targets ADAM19 to suppress proliferation, migration and invasion of retinoblastoma cells (80). ADAM19=A Disintegrin And Metalloproteinase 19

### ABCE1

Genistein upregulates miR-145 to target ABCE1 for suppressing retinoblastoma cell proliferation and inducing apoptosis (81). ATP-binding cassette sub-family E member 1 (ABCE1).

### CCPG1

miR-498 targets CCPG1 to upregulate retinoblastoma cell proliferation and inhibit cell apoptosis (82).

### DIXDC1

DIXDC1 appears to be a critical regulator for tumorigenesis by forming homomeric and heteromeric complexes with Axin and Dvl, two key mediators of Wnt signaling, to upregulate TCF-dependent transcription in Wnt signaling. (83-86). miR-186 can target DIXDC1 to inhibit cell proliferation and invasion of retinoblastoma cells (87)

### Runx3

miR-106b targets Runt-related transcription factor 3 (Runx3) to promote cell proliferation and migration (88).

### Pyruvate Dehydrogenase Kinase 1

PDK1 is upregulated in retinoblastoma cell lines and miR-138-5p can target PDK1 to inhibit cell migration and invasion and upregulate apoptosis in retinoblastoma cells (89, 90).

### Metadherin

miR-874 targets metadherin to promote cellular proliferation and invasion in retinoblastoma cells (91).

### CETN3

miR-410 targets CETN3 to promote cell proliferation, migration and invasion in retinoblastoma cells (92). Evidence also showed miR-410 is capable of activating the Wnt signaling pathway in retinoblastoma cells.

### CEMIP and CADM3

miR-140-5P appears to target cell adhesion molecule 3 (CADM3) and cell migration-inducing protein (CEMIP) to downregulate cellular proliferation, migration and invasion of retinoblastoma cell (93).

### Unstated Target

miR-222 promotes promote cellular proliferation migration and invasion in retinoblastoma cells (94). Another article states miR-222 targets Rb1 to promote retinoblastoma cell proliferation (95).

Identification of miRNAs with Rb Tumorigenesis by Microarray

| | | | | |
|---|---|---|---|---|
| miR-494, let-7e, miR-513-1, miR-513-2, miR- | upregulated | microarray | 9 | 100 |
| 518c, miR-129-1, miR-129-2, miR-198, miR-492, | | | | |
| miR-498, miR-320, miR-503, miR-373 | | | | |

No miRNA found downregulated with a change of more than twofold (96).

Differentially Expressed miRNAs in Retinoblastoma

| | | | | | |
|---|---|---|---|---|---|
| miR-129-3p, miR-382, miR-504, miR-22, miR- | downregulated | microarray | 12 | 100 | 106 |
| 874, miR-139-3p, miR-758, miR-655, miR-129- | | | | | |
| 5p, miR-200a, miR-370, miR-485-5p, miR-193a- | | | | | |
| 5p, miR-330-5p, miR-429, miR-889, miR-499-5p, | | | | | |
| miR-342-5p, miR-448, miR-200b, miR-196b, | | | | | |
| miR-518f, miR-34c-5p | | | | | |
| miR-138, miR-155, miR-106b, miR-216a, miR- | upregulated | microarray | 12 | 100 | 106 |
| 217, miR-20b, miR-17, miR-106a, miR-25, miR- | | | | | |
| 652, miR-301b, miR-886-5p, miR-93, miR-34a, | | | | | |
| miR-18a, miR-449a, miR-449b, miR-224 | | | | | |

Validated miRNAs and Associated Target Genes in Retinoblastoma

| | | | | | | | |
|---|---|---|---|---|---|---|---|
| miR-129-3p, | downregulated | 12 (plus | 100 | CDK4 and CDK6 | NA | no significant | 106 |
| miR-129-5p, | | 2 cell | | (miR-129); | | correlation of | |
| miR-382, | | lines and | | MYC | | miRNA expression | |
| miR-504, | | mouse | | (miR-382); | | and optic nerve | |
| miR-22 | | tumours) | | TP53 | | invasion or | |
| | | | | (miR-504); | | intraocular | |
| | | | | HDAC4and MYCP | | neovascularization | |
| | | | | (miR-22) | | | |

miRNAs in Rb Upregulated in Hypoxia

Hypoxic conditions in retinoblastoma upregulate miR-181b, miR-125a-3p and miR-30c-2 while downregulate miR-497 and miR-491-3p (97).

### COX-2/PGE2

miR-137 targets COX-2 and inhibits PGE2 synthesis to downregulate cell proliferation and invasion in retinoblastoma cells (98).

miRNome Landscape Analysis

Castro-Magdonel et al. proposed 30 miRNAs present in each of their 12 retinoblastoma tumor samples represent a common miRNA expression profile; highlighting miR-3613 because it potentially targets at least 36 tumor suppressor genes, including Rb1 (99).

miR-34b/c

Carvalho et al. proposed a signal nucleotide polymorphism (rs4938723T>C) in the miR-34B/C gene does not change the risk for retinoblastoma (100). They reported hereditary patients with retinoblastoma miR-34 b/c with the SNP CC genotype had a mean age at diagnosis lower (1.4+/−1.4 months) than patients with the retinoblastoma miR-34 b/c TT genotype (13.8+/−6.1 p=0.001). Screening for the mir-34b/c rs4938723T>C might prove useful as a biomarker for hereditary RB.

### Retinoblastoma Serum Biomarker of Group D and E Eyes

Beta et al. found 25 upregulated and 8 downregulated miRNAs in both serum and retinoblastoma tumors from their 14 Group D and E retinoblastoma patient cohort (101). rtPCR of 20 additional retinoblastoma serum sample reinforced three upregulated miRNAs (miR-17, miR-18a and miR-20a) and two downregulated (miR-19b and miR-92a-1). miRNA signature identification of retinoblastoma

Yang et Mei analyzed three retinoblastoma and three healthy retina samples to computationally identify the following miRNAs in retinoblastoma and their potential mechanisms: hsa-miR-373: RB invasion and metastasis. hsa-miR-125b and hsa-let-7b tumor suppressors via the coregulation of CDK6, CDC25A, and LIN28A, which all mediated the cell cycle pathway. hsa-miR-181a might involve in the CDKN1B-regulated cell cycle pathway. hsa-miR-25, hsa-miR-18a, and hsa-miR-20a coregulation of BCL2L1.

E2 Protein and miRNA Dysregulation in RB

E2F1 and E2F3 likely upregulate the miR-17-92 family in retinoblastoma cells as loss of either E2F1 or E2F3 resulted in wildtype levels of the miR-17-92 family (102). Other miRNA's and their upregulation/downregulation in response to the presence/absence of E2F1 and E2F3 are shown below:

Rb-mediated miRNA deregulation at P21 is rescued by loss of E2F1 and E2F3

| | | Expression compared | |
|---|---|---|---|
| | | to wt retina | |
| miRNA | Rb/p107 | Rb/p107/E2F1 | Rb/p107/E2F3 |
| mir-15a | down | no change | no change |
| mir-17 | up | no change | no change |
| mir-18a | up | no change | no change |
| mir-20a | up | no change | no change |
| mir-20b | up | no change | no change |
| mir-22 | down | no change | no change |
| mir-26b | down | no change | no change |
| mir-29cstar | down | no change | no change |
| mir-30bstar | down | no change | no change |
| mir-34b-3p | up | no change | no change |
| mir-34c | up | no change | no change |
| mir-93 | up | no change | no change |
| mir-96 | down | no change | no change |
| mir-106a | up | no change | no change |
| mir-124a(TM1182) | down | no change | no change |
| mir-124star | down | down | down |
| mir-142-3p | up | up | up |
| mir-143 | down | no change | no change |
| mir-145 | down | no change | no change |
| mir-146a | up | no change | no change |
| mir-146b | up | no change | no change |
| mir-182 | down | no change | no change |
| mir-183 | down | down | down |
| mir-183star | down | no change | no change |
| mir-335star | down | no change | no change |
| mir-378(TM2243) | down | no change | no change |
| mir-378(TM567) | down | no change | no change |
| mir-449b(TM1667) | up | no change | no change |
| mir-672 | up | no change | no change |
| mir-872star | down | no change | no change |

miRNA libraries can be built from the AH and to evaluate patterns; and specific miRNAs can be targeted as part of the analysis. An example is given below of 7 AH samples, a sample of AH from a patient with glaucoma as a control, and blood samples with miRNA expression.

| | RB_AH1 | RB_AH2 | RB_AH3 | RB_AH4 | RB_AH5 | RB_AH6 | RB_AH7 |
|---|---|---|---|---|---|---|---|
| miRNA-125b | 316 | 551 | 94 | 170 | 96 | 166 | 870 |
| miRNA-124 | 10 | 18 | 16 | 17 | 43 | 43 | 56 |
| miRNA-141 | 156 | 88 | 64 | 166 | 148 | 242 | 155 |
| miRNA-146a | 34 | 145 | 32 | 27 | 23 | 48 | 72 |
| miRNA-16 | 125 | 76 | 23 | 35 | 18 | 38 | 460 |
| miRNA-223 | 11 | 8 | 14 | 16 | 17 | 10 | 74 |
| miRNA-92a | 162 | 47 | 86 | 62 | 47 | 47 | 89 |
| miRNA-184 | 4584 | 145 | 163 | 334 | 70 | 205 | 8414 |

| | | | | level in | info in |
|---|---|---|---|---|---|
| | Glc_AH-1 | Blood 1 | Blood 2 | AH to blood | literature |
| miRNA-125b | 27 | 26 | 37 | upregulated | promotes |
| | | | | | proliferation |
| | | | | | and migration |
| miRNA-124 | 22 | 571 | 180 | downregulated | inhibits |
| | | | | | proliferation |
| | | | | | and invasion |
| miRNA-141 | 82 | 115 | 91 | normal to | can regulate |
| | | | | slightly | the RB1 pathway, |
| | | | | upregulated | has been described |
| | | | | | as up at |
| miRNA-146a | 38 | 173 | 46 | normal/ | involved in |
| | | | | variable | tumorigenesis |
| | | | | | and inflammation |
| miRNA-16 | 25 | 8,468 | 1,997 | downregulated | involved in |
| | | | | | other cancers |
| | | | | | with normal RB |
| miRNA-223 | 17 | 4,680 | 682 | downregulated | involved in sarcoma, |
| | | | | | which is mediated |
| | | | | | by RB1 |
| miRNA-92a | 56 | 93 | 30 | normal to | cooperative miRNA |
| | | | | slightly | complex 17-92 |
| | | | | upregulated | to promote |
| | | | | | retinoblast |
| miRNA-184 | 233 | 47 | 22 | upregulated | plays a role |
| | | | | | in multiple |
| | | | | | eye disorders |

### BIBLIOGRAPHY

Delsin, L. E. A., Salomao, K. B., Pezuk, J. A. et al. J Cancer Res Clin Oncol (2019) 145: 1. https://doi.org/10.1007/s00432-018-2773-7
Reis A H, Vargas F R, Lemos B. More epigenetic hits than meets the eye: microRNAs and genes associated with the tumorigenesis of retinoblastoma. Front Genet. 2012:3:284. Published 2012 Dec. 7. doi:10.3389/fgene.2012.00284
1. Lambertz I, Nittner D, Mestdagh P, et al. Monoallelic but not biallelic loss of Dicer1 promotes tumorigenesis in vivo. Cell Death Differ 2010; 17(4):633-41.
2. Wei J S, Song Y K, Durinck S, et al. The MYCN oncogene is a direct target of miR-34a. Oncogene 2008; 27(39):5204-13.
3. Buechner J, Tomte E, Haug B H, et al. Tumour-suppressor microRNAs let-7 and mir-101 target the proto-oncogene MYCN and inhibit cell proliferation in MYCN-amplified neuroblastoma. Br J Cancer 2011; 105(2):296-303.
4. Yan Su S L, Jincun Lic and Liya Deng. Shikonin-mediated up-regulation of miR-34a and miR-202 inhibits retinoblastoma proliferation. Toxicology Research 2018.
5. Fontana L, Fiori M E, Albini S, et al. Antagomir-17-5p abolishes the growth of therapy-resistant neuroblastoma through p21 and BIM. PLoS One 2008; 3(5):e2236.
6. Loven J, Zinin N, Wahlstrom T, et al. MYCN-regulated microRNAs repress estrogen receptor-alpha (ESRI) expression and neuronal differentiation in human neuroblastoma. Proc Natl Acad Sci USA 2010; 107(4):1553-8.
7. Mestdagh P, Fredlund E, Pattyn F, et al. MYCN/c-MYC-induced microRNAs repress coding gene networks associated with poor outcome in MYCN/c-MYC-activated tumors. Oncogene 2010; 29(9):1394-404.
8. Northcott P A, Femandez-L A, Hagan J P, et al. The miR-17/92 polycistron is up-regulated in sonic hedgehog-driven medulloblastomas and induced by N-myc in sonic hedgehog-treated cerebellar neural precursors. Cancer Res 2009; 69(8):3249-55.
9. Wu N, Jia D, Bates B, et al. A mouse model of MYCN-driven retinoblastoma reveals MYCN-independent tumor reemergence. J Clin Invest 2017; 127(3):888-98.
10. Li J, Zhang Y, Wang X, Zhao R. microRNA-497 overexpression decreases proliferation, migration and invasion of human retinoblastoma cells via targeting vascular endothelial growth factor A. Oncol Lett 2017; 13(6):5021-7.
11. Park S M, Kim K, Lee E J, et al. Reduced expression of DRAM2/TMEM77 in tumor cells interferes with cell death. Biochem Biophys Res Commun 2009; 390(4):1340-4.
12. Bai S, Tian B, Li A, et al. MicroRNA-125b promotes tumor growth and suppresses apoptosis by targeting DRAM2 in retinoblastoma. Eye (Lond) 2016; 30(12):1630-8.
13. Huang J, Yang Y, Fang F, Liu K. MALAT1 modulates the autophagy of retinoblastoma cell through miR-124-mediated stx17 regulation. J Cell Biochem 2018; 119(5):3853-63.
14. Hamasaki M, Furuta N, Matsuda A, et al. Autophagosomes form at E R-mitochondria contact sites. Nature 2013; 495(7441):389-93.
15. Liang Y, Chen X, Liang Z. MicroRNA-320 regulates autophagy in retinoblastoma by targeting hypoxia inducible factor-1α. Exp Ther Med 2017; 14(3):2367-72.
16. Liu S, Hu C, Wang Y, et al. miR-124 inhibits proliferation and invasion of human retinoblastoma cells by targeting STAT3. Oncol Rep 2016; 36(4):2398-404.
17. Liu S, Zhang X, Hu C, et al. miR-29a inhibits human retinoblastoma progression by targeting STAT3. Oncol Rep 2018; 39(2):739-46.
18. Jo D H, Kim J H, Cho C S, et al. STAT3 inhibition suppresses proliferation of retinoblastoma through down-regulation of positive feedback loop of STAT3/miR-17-92 clusters. Oncotarget 2014; 5(22):11513-25.
19. Vandhana S, Deepa P R, Jayanthi U, et al. Clinico-pathological correlations of fatty acid synthase expression in retinoblastoma: an Indian cohort study. Exp Mol Pathol 2011; 90(1):29-37.
20. Zhang J, Benavente C A, McEvoy J, et al. A novel retinoblastoma therapy from genomic and epigenetic analyses. Nature 2012; 481(7381):329-34.
21. Venkatesan N, Deepa P R, Khetan V, Krishnakumar S. Computational and in vitro Investigation of miRNA-Gene Regulations in Retinoblastoma Pathogenesis: miRNA Mimics Strategy. Bioinform Biol Insights 2015; 9:89-101.
22. Li X, Yang L, Shuai T, et al. MiR-433 inhibits retinoblastoma malignancy by suppressing Notch1 and PAX6 expression. Biomed Pharmacother 2016; 82:247-55.
23. Zhang M, Li Q, Pan Y, et al. MicroRNA-655 attenuates the malignant biological behaviours of retinoblastoma cells by directly targeting PAX6 and suppressing the ERK and p38 MAPK signalling pathways. Oncol Rep 2018; 39(4):2040-50.
24. Wang J, Wang X, Wu G, et al. MiR-365b-3p, down-regulated in retinoblastoma, regulates cell cycle progression and apoptosis of human retinoblastoma cells by targeting PAX6. FEBS Lett 2013; 587(12):1779-86.
25. Li J, You X. MicroRNA-758 inhibits malignant progression of retinoblastoma by directly targeting PAX6. Oncol Rep 2018; 40(3):1777-86.
26. Marine J C, Jochemsen A G. Mdmx and Mdm2: brothers in arms? Cell Cycle 2004; 3(7):900-4.
27. Komori H, Enomoto M, Nakamura M, et al. Distinct E2F-mediated transcriptional program regulates p14ARF gene expression. EMBO J 2005; 24(21):3724-36.
28. Sherr C J. The INK4a/ARF network in tumour suppression. Nat Rev Mol Cell Biol 2001; 2(10):731-7.
29. Sherr C J. Divorcing ARF and p53: an unsettled case. Nat Rev Cancer 2006; 6(9):663-73.
30. Sherr C J, Weber J D. The ARF/p53 pathway. Curr Opin Genet Dev 2000; 10(1):94-9.
31. To K H, Pajovic S, Gallie B L, Theriault B L. Regulation of p14ARF expression by miR-24: a potential mechanism compromising the p53 response during retinoblastoma development. BMC Cancer 2012; 12:69.
32. Nittner D, Lambertz I, Clermont F, et al. Synthetic lethality between Rb, p53 and Dicer or miR-17-92 in retinal progenitors suppresses retinoblastoma formation. Nat Cell Biol 2012; 14(9):958-65.
33. Jo D H, Kim J H, Park W Y, et al. Differential profiles of microRNAs in retinoblastoma cell lines of different proliferation and adherence patterns. J Pediatr Hematol Oncol 2011; 33(7):529-33.
34. McEvoy J, Ulyanov A, Brennan R, et al. Analysis of MDM2 and MDM4 single nucleotide polymorphisms, mRNA splicing and protein expression in retinoblastoma. PLoS One 2012; 7(8):e42739.
35. Liu Y, Li H, Zhu Z. MiR-22-3p targeting alpha-enolase 1 regulates the proliferation of retinoblastoma cells. Biomed Pharmacother 2018; 105:805-12.
36. Sreenivasan S, Thirumalai K, Danda R, Krishnakumar S. Effect of curcumin on miRNA expression in human Y79 retinoblastoma cells. Curr Eye Res 2012; 37(5):421-8.
37. Sun M, Estrov Z, Ji Y, et al. Curcumin (diferuloylmethane) alters the expression profiles of microRNAs in human pancreatic cancer cells. Mol Cancer Ther 2008; 7(3):464-73.
38. Dong H, Chen L. B7-H1 pathway and its role in the evasion of tumor immunity. J Mol Med (Berl) 2003; 81(5):281-7.
39. Wu L, Chen Z, Zhang J, Xing Y. Effect of miR-513a-5p on etoposide-stimulating B7-H1 expression in retinoblastoma cells. J Huazhong Univ Sci Technolog Med Sci 2012; 32(4):601-6.
40. Li M, Chen X M, Wang D M, et al. Effects of miR-26a on the expression of Beclin 1 in retinoblastoma cells. Genet Mol Res 2016; 15(2).
41. Zhang Y, Wu J H, Han F, et al. Arsenic trioxide induced apoptosis in retinoblastoma cells by abnormal expression of microRNA-376a. Neoplasma 2013; 60(3):247-53.
42. Lize M, Pilarski S, Dobbelstein M. E2F1-inducible microRNA 449a/b suppresses cell proliferation and promotes apoptosis. Cell Death Differ 2010; 17(3):452-8.
43. Yang X, Feng M, Jiang X, et al. miR-449a and miR-449b are direct transcriptional targets of E2F1 and negatively regulate pRb-E2F1 activity through a feedback loop by targeting CDK6 and CDC25A. Genes Dev 2009; 23(20):2388-93.
44. Martin A, Jones A, Bryar P J, et al. MicroRNAs-449a and -449b exhibit tumor suppressive effects in retinoblastoma. Biochem Biophys Res Commun 2013; 440(4):599-603.
45. Zhang Y, Zhu X, Wu Y, et al. MiR-613 suppresses retinoblastoma cell proliferation, invasion, and tumor formation by targeting E2F5. Tumour Biol 2017; 39(3):1010428317691674.
46. Mu G, Liu H, Zhou F, et al. Correlation of overexpression of HMGA1 and HMGA2 with poor tumor differentiation, invasion, and proliferation associated with let-7 down-regulation in retinoblastomas. Hum Pathol 2010; 41(4):493-502.
47. Venkatesan N, Deepa P, Vasudevan M, et al. Integrated Analysis of Dysregulated miRNA-gene Expression in HMGA2-silenced Retinoblastoma Cells. Bioinform Biol Insights 2014; 8:177-91.
48. Venkatesan N, Krishnakumar S, Deepa P R, et al. Molecular deregulation induced by silencing of the high mobility group protein A2 gene in retinoblastoma cells. Mol Vis 2012; 18:2420-37.
49. Huang J C, Babak T, Corson T W, et al. Using expression profiling data to identify human microRNA targets. Nat Methods 2007; 4(12):1045-9.
50. Wu X, Zeng Y, Wu S, et al. MiR-204, down-regulated in retinoblastoma, regulates proliferation and invasion of human retinoblastoma cells by targeting CyclinD2 and MMP-9. FEBS Lett 2015; 589(5):645-50.
51. Wang J X, Yang Y, Li K. Long noncoding RNA DANCR aggravates retinoblastoma through miR-34c and miR-613 by targeting MMP-9. J Cell Physiol 2018; 233(10):6986-95.
52. Zhang Y, Xue C, Zhu X, et al. Suppression of microRNA-125a-5p upregulates the TAZ-EGFR signaling pathway and promotes retinoblastoma proliferation. Cell Signal 2016; 28(8):850-60.
53. Jia M, Wei Z, Liu P, Zhao X. Silencing of ABCG2 by MicroRNA-3163 Inhibits Multidrug Resistance in Retinoblastoma Cancer Stem Cells. J Korean Med Sci 2016; 31(6):836-42.
54. Shao X L, Chen Y, Gao L. MiR-200c suppresses the migration of retinoblastoma cells by reversing epithelial mesenchymal transition. Int J Ophthalmol 2017; 10(8):1195-202.
55. Yang G, Fu Y, Lu X, et al. LncRNA HOTAIR/miR-613/c-met axis modulated epithelial-mesenchymal transition of retinoblastoma cells. J Cell Mol Med 2018.
56. Ding Y, Wu M, Liu J, et al. Seed-targeting anti-miR-21 inhibiting malignant progression of retinoblastoma and analysis of their phosphorylation signaling pathways. Exp Eye Res 2014; 122:1-8.
57. Shen F, Mo M H, Chen L, et al. MicroRNA-21 Down-regulates Rb1 Expression by Targeting PDCD4 in Retinoblastoma. J Cancer 2014; 5(9):804-12.
58. Gui F, Hong Z, You Z, et al. MiR-21 inhibitor suppressed the progression of retinoblastoma via the modulation of PTEN/PI3K/AKT pathway. Cell Biol Int 2016; 40(12):1294-302.
59. Chen DSJDYYY. MicroRNA 382 inhibits cell proliferation and invasion of retinoblastoma by targeting BDNF mediated PI3K/AKT signaling pathway. 2017.
60. Wei D, Miao Y, Yu L, et al. Downregulation of microRNA-198 suppresses cell proliferation and invasion in retinoblastoma by directly targeting PTEN. Mol Med Rep 2018; 18(1):595-602.
61. Wu S, Ai N, Liu Q, Zhang J. MicroRNA-448 inhibits the progression of retinoblastoma by directly targeting ROCK1 and regulating PI3K/AKT signalling pathway. Oncol Rep 2018; 39(5):2402-12.
62. Xu X, Ge S, Jia R, et al. Hypoxia-induced miR-181b enhances angiogenesis of retinoblastoma cells by targeting PDCD10 and GATA6. Oncol Rep 2015; 33(6):2789-96.
63. Beta M, Khetan V, Chatterjee N, et al. EpCAM knockdown alters microRNA expression in retinoblastoma—functional implication of EpCAM regulated miRNA in tumor progression. PLoS One 2014; 9(12):e114800.
64. Kandalam M M, Beta M, Maheswari U K, et al. Oncogenic microRNA 17-92 cluster is regulated by epithelial cell adhesion molecule and could be a potential therapeutic target in retinoblastoma. Mol Vis 2012; 18:2279-87.
65. H E QJW, CHEN L, YANG Y, et al. MicroRNA-101-3p inhibits proliferation in retinoblastoma cells
by targeting EZH2 and HDAC9. EXPERIMENTAL AND THERAPEUTIC MEDICINE 2018.
66. Bracken A P, Pasini D, Capra M, et al. EZH2 is downstream of the pRB-E2F pathway, essential for proliferation and amplified in cancer. EMBO J 2003; 22(20):5323-35.
67. Varambally S, Dhanasekaran S M, Zhou M, et al. The polycomb group protein EZH2 is involved in progression of prostate cancer. Nature 2002; 419(6907):624-9.
68. Lei Q, Shen F, Wu J, et al. MiR-101, downregulated in retinoblastoma, functions as a tumor suppressor in human retinoblastoma cells by targeting EZH2. Oncol Rep 2014; 32(1):261-9.
69. Dalgard C L, Gonzalez M, deNiro J E, O'Brien J M. Differential microRNA-34a expression and tumor suppressor function in retinoblastoma cells. Invest Ophthalmol Vis Sci 2009; 50(10):4542-51.
70. Liu K, Huang J, Xie M, et al. MIR34A regulates autophagy and apoptosis by targeting HMGB1 in the retinoblastoma cell. Autophagy 2014; 10(3):442-52.
71. Wang J, Wang X, Li Z, et al. MicroRNA-183 suppresses retinoblastoma cell growth, invasion and migration by targeting LRP6. FEBS J 2014; 281(5):1355-65.
72. Conkrite K, Sundby M, Mukai S, et al. miR-17-92 cooperates with R B pathway mutations to promote retinoblastoma. Genes Dev 2011; 25(16):1734-45.
73. Subramanian N, Kanwar J R, Kanwar R K, Krishnakumar S. Blocking the maturation of OncomiRNAs using pri-miRNA-17-92 aptamer in retinoblastoma. Nucleic Acid Ther 2015; 25(1):47-52.
74. Zhang A, Shang W, Nie Q, et al. Long non-coding RNA H19 suppresses retinoblastoma progression via counteracting miR-17-92 cluster. J Cell Biochem 2018; 119(4):3497-509.
75. Chen Y Z, Liu Z P, Zhou K Y, Li B. [Value of serum miR-17-92 cluster in diagnosis of retinoblastoma]. Zhongguo Dang Dai Er Ke Za Zhi 2017; 19(7):776-80.
76. Bindu Salim1* SMD, Madhu Beta2, Janakiraman Narayanan2, Subramanian Krishnakumar2 and Thalakkotur Lazar Mathew1. PMMA Platform Based Micro Fluidic Mixer for the Detection of MicroRNA-18a from Retinoblastoma Serum. 2015.
77. Wang L L, Hu H F, Feng Y Q. Suppressive effect of microRNA-143 in retinoblastoma. Int J Ophthalmol 2016; 9(11):1584-90.
78. Fadakar P, Akbari A, Ghassemi F, et al. Evaluation of SD-208, a TGF-β-RI Kinase Inhibitor, as an Anticancer Agent in Retinoblastoma. Acta Med Iran 2016; 54(6):352-8.
79. Zhao Y, Zhang S, Zhang Y. MicroRNA-320 inhibits cell proliferation, migration and invasion in retinoblastoma by targeting specificity protein 1. Mol Med Rep 2017; 16(2):2191-8.
80. Sun Z, Zhang A, Jiang T, et al. MiR-145 suppressed human retinoblastoma cell proliferation and invasion by targeting ADAM19. Int J Clin Exp Pathol 2015; 8(11):14521-7.
81. Wei D, Yang L, Lv B, Chen L. Genistein suppresses retinoblastoma cell viability and growth and induces apoptosis by upregulating miR-145 and inhibiting its target ABCE1. Mol Vis 2017; 23:385-94.
82. Yang L, Wei N, Wang L, et al. miR-498 promotes cell proliferation and inhibits cell apoptosis in retinoblastoma by directly targeting CCPG1. Childs Nerv Syst 2018; 34(3):417-22.
83. Yan X, Lyu T, Jia N, et al. Huaier aqueous extract inhibits ovarian cancer cell motility via the AKT/GSK30/0-catenin pathway. PLoS One 2013; 8(5):e63731.
84. Wang L, Cao X X, Chen Q, et al. DIXDC1 targets p21 and cyclin D1 via PI3K pathway activation to promote colon cancer cell proliferation. Cancer Sci 2009; 100(10):1801-8.
85. Schwarz-Romond T, Fiedler M, Shibata N, et al. The DIX domain of Dishevelled confers Wnt signaling by dynamic polymerization. Nat Struct Mol Biol 2007; 14(6):484-92.
86. Shiomi K, Uchida H, Keino-Masu K, Masu M. Ccdl, a novel protein with a DIX domain, is a positive regulator in the Wnt signaling during zebrafish neural patterning. Curr Biol 2003; 13(1):73-7.
87. Che X, Qian Y, Li D. Suppression of Disheveled-Axin Domain Containing 1 (DIXDC1) by MicroRNA-186 Inhibits the Proliferation and Invasion of Retinoblastoma Cells. J Mol Neurosci 2018; 64(2):252-61.
88. Yang G, Fu Y, Zhang L, et al. miR106b regulates retinoblastoma Y79 cells through Runx3. Oncol Rep 2017; 38(5):3039-43.
89. Wang Z, Yao Y J, Zheng F, et al. Mir-138-5p acts as a tumor suppressor by targeting pyruvate dehydrogenase kinase 1 in human retinoblastoma. Eur Rev Med Pharmacol Sci 2017; 21(24):5624-9.
90. Sradhanjali S, Tripathy D, Rath S, et al. Overexpression of pyruvate dehydrogenase kinase 1 in retinoblastoma: A potential therapeutic opportunity for targeting vitreous seeds and hypoxic regions. PLoS One 2017; 12(5):e0177744.
91. Zhang Y, Wang X, Zhao Y. MicroRNA-874 prohibits the proliferation and invasion of retinoblastoma cells by directly targeting metadherin. Mol Med Rep 2018; 18(3):3099-105.
92. Xiaohui Fan C Y, Shuxia Fan. miR-410 promotes the proliferation and migration in retinoblastoma cells via CETN3. Int J Clin Exp Pathol 2017.
93. Miao X, Wang Z, Chen B, et al. miR-140-5p suppresses retinoblastoma cell proliferation, migration, and invasion by targeting CEMIP and CADM3. Cell Mol Biol (Noisy-le-grand) 2018; 64(6):42-7.
94. Liu H, Cao B, Zhao Y, et al. Upregulated miR-221/222 promotes cell proliferation and invasion and is associated with invasive features in retinoblastoma. Cancer Biomark 2018.
95. LIU Yuefeng Z Y, ZHONG Xiaodong, LUO Weimin. miR-222 promotes retinoblastoma cell proliferation and invasion by targeting RB1. China Oncology 2016.
96. Zhao J J, Yang J, Lin J, et al. Identification of miRNAs associated with tumorigenesis of retinoblastoma by miRNA microarray analysis. Childs Nerv Syst 2009; 25(1):13-20.
97. Xu X, Jia R, Zhou Y, et al. Microarray-based analysis: identification of hypoxia-regulated microRNAs in retinoblastoma cells. Int J Oncol 2011; 38(5):1385-93.
98. Zhang J, He J, Zhang L. The down-regulation of microRNA-137 contributes to the up-regulation of retinoblastoma cell proliferation and invasion by regulating COX-2/PGE2 signaling. Biomed Pharmacother 2018; 106:35-42.
99. Castro-Magdonel B E, Orjuela M, Camacho J, et al. miRNome landscape analysis reveals a 30 miRNA core in retinoblastoma. BMC Cancer 2017; 17(1):458.
100. Carvalho I N, Reis A H, Dos Santos A C, Vargas F R. A polymorphism in mir-34b/c as a potential biomarker for early onset of hereditary retinoblastoma. Cancer Biomark 2017; 18(3):313-7.
101. Beta M, Venkatesan N, Vasudevan M, et al. Identification and Insilico Analysis of Retinoblastoma Serum microRNA Profile and Gene Targets Towards Prediction of Novel Serum Biomarkers. Bioinform Biol Insights 2013; 7:21-34.
102. Benavente C A, Finkelstein D, Johnson D A, et al. Chromatin remodelers HELLS and UHRF1 mediate the epigenetic deregulation of genes that drive retinoblastoma tumor progression. Oncotarget 2014; 5(20):9594-608.
miRNA-125b https://www.nature.com/articles/eye2016189
miRNA-124 https://www.ncbi.nlm.nih.gov/pubmed/27498908
miRNA-141 https://www.karger.com/Article/Fulltext/438641
miRNA-146a https://www.ncbi.nlm.nih.gov/pmc/articles/PMC3516829/
miRNA-16 https://www.sciencedirect.com/topics/neuroscience/microma-16
miRNA-223 https://www.ncbi.nlm.nih.gov/pubmed/22627383
miRNA-92a https://www.ncbi.nlm.nih.gov/pmc/articles/PMC3165937/
miRNA-184 https://www.karger.com/Article/PDF/371853

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of”

The articles “a” and “an” are used herein to refer to one or to more than one (i.e. to at least one) of the grammatical object of the article. By way of example, “an element” means one element or more than one element.

“Plurality” means at least two.

A “subject” or “patient” is a vertebrate, including a mammal, such as a human. Mammals include, but are not limited to, humans, farm animals, sport animals and pets.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 20% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

The term “gene” refers to a nucleic acid sequence that comprises control and coding sequences necessary for producing a polypeptide or precursor. The polypeptide may be encoded by a full-length coding sequence or by any portion of the coding sequence. The gene may be derived in whole or in part from any source known to the art, including a plant, a fungus, an animal, a bacterial genome or episome, eukaryotic, nuclear or plasmid DNA, cDNA, viral DNA, or chemically synthesized DNA. A gene may contain one or more modifications in either the coding or the untranslated regions that could affect the biological activity or the chemical structure of the expression product, the rate of expression, or the manner of expression control. Such modifications include, but are not limited to, mutations, insertions, deletions, and substitutions of one or more nucleotides. The gene may constitute an uninterrupted coding sequence, or it may include one or more introns, bound by the appropriate splice junctions.

The term “gene expression” refers to the process by which a nucleic acid sequence undergoes successful transcription and/or translation such that detectable levels of the nucleotide sequence are expressed.

The terms “gene expression profile” or “gene signature” refer to a group of genes expressed by a particular cell or tissue type wherein presence of the genes taken together or the differential expression of such genes, is indicative/predictive of a certain condition.

The term “nucleic acid” as used herein, refers to a molecule comprised of one or more nucleotides, i.e., ribonucleotides, deoxyribonucleotides, or both. The term includes monomers and polymers of ribonucleotides and deoxyribonucleotides, with the ribonucleotides and/or deoxyribonucleotides being bound together, in the case of the polymers, via 5′ to 3′ linkages. The ribonucleotide and deoxyribonucleotide polymers may be single or double-stranded. However, linkages may include any of the linkages known in the art including, for example, nucleic acids comprising 5′ to 3′ linkages. Furthermore, the term “nucleic acid sequences” contemplates the complementary sequence and specifically includes any nucleic acid sequence that is substantially homologous to the both the nucleic acid sequence and its complement.

The terms “array” and “microarray” refer to the type of genes represented on an array by oligonucleotides, and where the type of genes represented on the array is dependent on the intended purpose of the array (e.g., to monitor expression of human genes). The oligonucleotides on a given array may correspond to the same type, category, or group of genes. Genes may be considered to be of the same type if they share some common characteristics such as species of origin (e.g., human, mouse, rat); disease state (e.g., cancer); functions (e.g., protein kinases, tumor suppressors); or same biological process (e.g., apoptosis, signal transduction, cell cycle regulation, proliferation, differentiation). For example, one array type may be a “cancer array” in which each of the array oligonucleotides correspond to a gene associated with a cancer.

The term “activation” as used herein refers to any alteration of a signaling pathway or biological response including, for example, increases above basal levels, restoration to basal levels from an inhibited state, and stimulation of the pathway above basal levels.

The term “differential expression” refers to both quantitative as well as qualitative differences in the temporal and tissue expression patterns of a gene in diseased tissues or cells versus normal adjacent tissue. For example, a differentially expressed gene may have its expression activated or partially or completely inactivated in normal versus disease conditions or may be up-regulated (over-expressed) or down-regulated (under-expressed) in a disease condition versus a normal condition. Such a qualitatively regulated gene may exhibit an expression pattern within a given tissue or cell type that is detectable in either control or disease conditions but is not detectable in both. Stated another way, a gene is differentially expressed when expression of the gene occurs at a higher or lower level in the diseased tissues or cells of a patient relative to the level of its expression in the normal (disease-free) tissues or cells of the patient and/or control tissues or cells.

The term “biological sample” refers to a sample obtained from an organism (e.g., a human patient) or from components (e.g., cells) of an organism. The sample may be of any biological tissue or fluid. The sample may be a “clinical sample” which is a sample derived from a patient. Such samples include, but are not limited to, sputum, blood, blood cells (e.g., white cells), amniotic fluid, plasma, semen, bone marrow, circulating tumor cells, circulating DNA, circulating exosomes, and tissue or fine needle biopsy samples, urine, peritoneal fluid, aqueous humor, and pleural fluid, or cells therefrom. Biological samples may also include sections of tissues such as frozen sections or formalin fixed paraffin embedded sections akin for histological purposes. A biological sample may also be referred to as a “patient sample.”

As used herein, “health care provider” includes either an individual or an institution that provides preventive, curative, promotional or rehabilitative health care services to a subject, such as a patient. In one embodiment, the data is provided to a health care provider so that they may use it in their diagnosis/treatment of the patient.

The term “standard,” as used herein, refers to something used for comparison, such as control or a healthy subject.

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

What is claimed is:

1. A method of performing prognosis or diagnosis of retinoblastoma in a subject, comprising:
assaying cell-free DNA (cfDNA) or cell-free miRNA from aqueous humor of the subject for somatic chromosomal copy number alterations (SCNAs) or an increase or a decrease in miRNA, and
determining a high likelihood or severity of retinoblastoma if at least one chromosome has a gain in SCNA or at least one miRNA is increased or decreased compared to that of a control.

2. The method of claim 1, wherein the control is cfDNA or cell-free miRNA from aqueous humor of an eye that does not have retinoblastoma.

3. The method of claim 1, wherein the gain in SCNA of the at least one chromosome is statistically significantly higher, as characterized by p<0.05, than that of a control (for example between eyes of the same subject or comparing with an Rb free subject.

4. The method of claim 1, wherein the at least one chromosome having a gain comprises chromosome 6p.

5. The method of claim 4, wherein chromosome 6p has a mean amplitude gain of at least >1.40 ratio to the median copy number gain, wherein the control has a mean amplitude gain of no more than 1.10 (such that the treated eyes are not considered controls, the point of the prognostication is that eyes that do well (salvaged) and eyes that are enucleated have a significant difference in 6p. Scientifically an eye that is a control does not have the disease—these are diseased eyes with 2 different outcomes).

6. The method of claim 1, wherein the at least one chromosome having a gain in SCNA is 1q or 2p.

7. The method of claim 1, wherein the cfDNA or cell-free miRNA is obtained from aqueous humor comprises tumor-derived cell-free DNA or miRNA.

8. The method of claim 1, wherein the cfDNA or miRNA from aqueous humor is taken after initial chemotherapy to treat retinoblastoma.

9. The method of claim 1, wherein the cfDNA or miRNA from aqueous humor is taken at the time or following a tumor recurrence.

10. The method of claim 1, wherein the assay comprises shallow whole genome sequencing.

11. A method of evaluating retinoblastoma response to a prior treatment and prescribing intervention in a subject in need thereof, comprising:
(i) assaying cell-free DNA (cfDNA) from aqueous humor of the eye having active or treated retinoblastoma of the subject for somatic chromosomal copy number alterations (SCNAs), and
(ii) determining poor retinoblastoma response to the prior treatment if chromosome 6p has a gain in SCNA of >1.40 ratio to the median copy number gain, these eyes likely require enucleation to treat the active disease; or determining positive retinoblastoma response to a prior treatment and excluding enucleation of the eye if chromosome 6p does not have a gain in SCNA or shows a response correlating with clinical response to chosen therapy.

12. The method of claim 11, wherein the prior treatment comprises chemotherapy, which is the foundation for all salvage regimens in retinoblastoma.

13. A method of monitoring progression of retinoblastoma in a subject in need thereof, comprising:
assaying cell-free DNA (cfDNA) from aqueous humor of the eye having or having had retinoblastoma of the subject for somatic chromosomal copy number alterations (SCNAs),
wherein an increase in the SCNAs over a period of time indicates a progression of the retinoblastoma; and a decrease in the presence of SCNA over a period of time/therapy indicates a positive treatment effect for retinoblastoma.

14. The method of claim 13, wherein the period of time begins before a therapeutic treatment and concludes after a therapeutic treatment.

15. The method of claim 13, wherein the period of time begins and concludes after a therapeutic treatment.

16. The method of claim 13, wherein the period of time begins after a first therapeutic treatment and before a second therapeutic treatment and concludes after a second therapeutic treatment.

17. A method of detecting somatic chromosomal copy number alterations (SCNAs) from cell-free DNA or an increase or decrease in cell-free miRNA, comprising:
assaying cell-free DNA (cfDNA) or miRNA from aqueous humor of the subject for somatic chromosomal copy number alterations (SCNAs).

18. The method of claim 17, wherein the SCNAs is a gain of chromosome 6p.

19. The method of any one of claims 1, 2, 7-10 or 17, wherein the miRNA is at least one of the miRNAs provided in FIG. 9.

20. The method of any one of claims 1, 2, 7-10, 17 or 20, wherein the increase or decrease of at least one miRNA is as depicted in FIG. 9.

21. The method of any one of claims 1-20, further comprising treating said subject for retinoblastoma comprising administering at least one of cryotherapy, thermotherapy, chemotherapy, radiation therapy, high-dose chemotherapy with stem cell rescue or surgery, including enucleation.
